(19) United States
(12) Patent Application Publication
(10) Pub. No.: US 2023/0178185 A1
(43) Pub. Date: Jun. 8, 2023

(54) METHODS AND SYSTEMS FOR STABILIZING PROTEINS USING INTELLIGENT AUTOMATION

(71) Applicant: Rutgers, The State University of New Jersey, New Brunswick, NJ (US)

(72) Inventors: Adam GORMLEY, New Brunswick, NJ (US); Matthew TAMASI, New Brunswick, NJ (US)

(21) Appl. No.: 17/922,938

(22) PCT Filed: May 6, 2021
(86) PCT No.: PCT/US21/31114

§ 371 Date: Nov. 2, 2022

Related U.S. Application Data
(60) Provisional application No. 63/021,711, filed on May 8, 2020.

(51) Int. Cl. G16B 40/00; G16B 15/00; G16C 20/64; G16C 20/50; G16C 20/70; G16C 20/30

(52) U.S. Cl. CPC G16B 40/00; G16B 15/00; G16C 20/64; G16C 20/50; G16C 20/70; G16C 20/30

(57) ABSTRACT

A method includes receiving one input feature and one output feature of importance of polymers used to stabilize a protein. A set of polymers from a library are identified based on the input feature and the output feature of importance that are applied to a machine learning model. Data for each polymer in the library includes features for each polymer and reagents for stabilizing the protein. Each polymer in the identified set is used to stabilize samples of the protein in well plates in a well plate array based on the reagents from the library data in the identified set. A score for each sample of the protein is assigned by comparing the measured output feature from the well plates corresponding to the identified set to the output feature of importance. The samples of the protein are identified having scores higher than a predefined threshold.

### RELATED APPLICATION

This patent application claims the benefit of and the priority to U.S. Provisional Patent Application Ser. No. 63/021,711, filed May 8, 2020, the entirety of which is incorporated by reference in its entirety.

### FIELD

The present disclosure relates to the design of proteins, and more particularly to methods and systems for stabilizing proteins using intelligent automation.

### BACKGROUND

Proteins may be used in many biopharmaceuticals, biological and medical applications. Biopharmaceuticals may include any pharmaceutical drug product manufactured in, extracted from, or synthesized in part from biological sources. Biopharmaceuticals may include, for example, vaccines, blood, blood components, allergenics, somatic cells, gene therapies, tissues, recombinant therapeutic protein, and living medicines used in cell therapy. They may be composed of sugars, proteins, or nucleic acids or complex combinations of these substances, or may be living cells or tissues.

Proteins, in the form of enzymes, play a significant role in many commercial and industrial due to their high catalytic potential across a wide range of substrates. For example, enzymes typically operate under precise conditions of temperature and pH. However, ex vivo conditions for using enzymes are more demanding. In most instances, these enzymes are exposed to harsh conditions such as organic solvents, heat, denaturants or acids/bases to facilitate process efficiency. However, these harsh conditions result in enzyme destabilization which necessitates continuous addition of fresh and costly enzyme to the reaction mixture.

Complex synthetic polymers may stabilize proteins such as enzymes under harsh conditions by providing a chaperone-like stabilizing shell. More recently, the use of single enzyme nanoparticles (SEN) has emerged as an attractive method for stabilizing enzymes, for example. In these cases, individual enzymes may be wrapped in a protective coating to stabilize the enzyme structure. By carefully designing this enzyme-material interface, it may be possible to provide enzyme durability in extremely unnatural environments during the polymer synthesis that the enzyme is catalyzing.

For example, a matching of polymer-protein characteristics (i.e. hydrophobicity, polarity, charge, etc.) which allowed for full horseradish peroxidase (HRP) enzyme activity in pure toluene was demonstrated. (See Panganiban et al., Science, vol. 359, pp. 1239-1243, March 2018). However, the demonstrated single enzyme nanoparticle (SEN) discovery process utilized molecular dynamic simulations and manual trial-and-error experimentation of polymers designs, which may be time consuming, and may likely miss key and unexpected physicochemical contributions that lead to stable complexes.

There is a need in the art for methods and systems for stabilizing proteins for preventing protein denaturation and prolonging enzyme durability.

### SUMMARY

In some embodiments, a method may include:

receiving, by a processor, from a user, at least one protein for stabilization using polymers, and at least one input feature and at least one output feature of importance of polymers used to stabilize the at least one protein;

identifying, by the processor, a set of polymers from a library of a plurality of polymers for stabilizing the at least one protein using an output of at least one machine learning model;

wherein the at least one machine learning model may output at least one predicted output feature for each polymer in the library corresponding to the at least one output feature of importance of polymers used to stabilize the at least one protein when inputting data for each polymer in the library into the at least one machine learning model;

wherein the data for each polymer in the library of the plurality of polymers may include at least:
(i) features for each polymer, and
(ii) reagents for stabilizing the at least one protein;

generating, by the processor, a controller script for implementing an experimental design flow for stabilizing samples of the at least one protein in a plurality of well plates in a well plate array based on the at least one predicted output feature for each polymer in the identified set;

wherein each sample of the at least one protein in the plurality of well plates in the well plate array may correspond to each polymer in the identified set of polymers;

wherein the controller script may be configured to control at least one instrument, at least one measurement device, or both in an instrumentation platform for:
(i) dispensing the at least one protein and reagents for stabilizing the at least one protein into each well plate in the well plate array;
(ii) initiating polymerization of the samples in each well plate; and
(iii) measuring the at least one output feature of the samples of the at least one protein in each well plate corresponding to the at least one output feature of importance;

executing, by the processor, the controller script for implementing the experimental design flow;

assigning, by the processor, a score to each sample of the at least one protein in the well plate array based on a comparison between the at least one measured output feature of the polymer in each well plate and the at least one output feature of importance;

wherein a higher score may be indicative of a higher match between the at least one measured output feature of the polymer and the at least one output feature of importance of the polymer used to stabilize the at least one protein; and

identifying, by the processor, the samples of the at least one protein in the well plate array with scores higher than a predefined threshold.

In some embodiments, a system may include an instrumentation platform including at least one instrument, at least one measurement device, or both, and at least one processor. The at least one processor may be configured to:

receive from a user, at least one protein for stabilization using polymers, and at least one input feature and at least one output feature of importance of polymers used to stabilize the at least one protein;

identify a set of polymers from a library of a plurality of polymers for stabilizing the at least one protein using an output of at least one machine learning model;

wherein the at least one machine learning model may output at least one predicted output feature for each polymer in the library corresponding to the at least one output feature of importance of polymers used to stabilize the at least one protein when inputting data for each polymer in the library into the at least one machine learning model;

wherein the data for each polymer in the library of the plurality of polymers may include at least:
(i) features for each polymer, and
(ii) reagents for stabilizing the at least one protein;

generate a controller script for implementing an experimental design flow for stabilizing samples of the at least one protein in a plurality of well plates in a well plate array based on the at least one predicted output feature for each polymer in the identified set;

wherein each sample of the at least one protein in the plurality of well plates in the well plate array may correspond to each polymer in the identified set of polymers;

wherein the controller script may be configured to control at least one instrument, at least one measurement device, or both in an instrumentation platform for:
(i) dispensing the at least one protein and reagents for stabilizing the at least one protein into each well plate in the well plate array;
(ii) initiating polymerization of the samples in each well plate; and
(iii) measuring the at least one output feature of the samples of the at least one protein in each well plate corresponding to the at least one output feature of importance;

execute the controller script for implementing the experimental design flow;

assign a score to each sample of the at least one protein in the well plate array based on a comparison between the at least one measured output feature of the polymer in each well plate and the at least one output feature of importance;

wherein a higher score may be indicative of a higher match between the at least one measured output feature of the polymer and the at least one output feature of importance of the polymer used to stabilize the at least one protein; and

identify the samples of the at least one protein in the well plate array with scores higher than a predefined threshold.

### DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 illustrates a flow diagram of a Design-Build-Test-Learn experimentation workflow for optimizing enzyme designs, in accordance with one or more embodiments of the present disclosure;

FIG. 2 illustrates a flow diagram of a fully autonomous controlled/living radical polymerizations (CLRP) flow for optimizing stable enzyme designs, in accordance with one or more embodiments of the present disclosure;

FIG. 3 illustrates a fully autonomous instrumentation platform for optimizing enzyme designs, in accordance with one or more embodiments of the present disclosure;

FIG. 4 illustrates a flow diagram of a machine learning guided approach for optimizing enzyme designs, in accordance with one or more embodiments of the present disclosure;

FIGS. 5A-5D are machine learning model-generated plots illustrating feature importance of four tested monomers, in accordance with one or more embodiments of the present disclosure;

FIGS. 6A-6D are graphs comparing a performance between Generation 1 (G1) versus Generation 2 (G2) polymer libraries and model analysis, in accordance with one or more embodiments of the present disclosure;

FIG. 7 is a table of five representative enzymes may also be used in an automated controlled/living radical polymerizations (CLRP) flow for polymer synthesis, in accordance with one or more embodiments of the present disclosure;

FIG. 8 shows acrylate monomers in a G1 polymer library database, in accordance with one or more embodiments of the present disclosure;

FIGS. 9A-9B are graphs of automated polymer synthesis conversion and a respective molecular weight distribution, in accordance with one or more embodiments of the present disclosure;

FIG. 10 is a flowchart of a method for measuring protective effects of polymers and enzyme denaturation, in accordance with one or more embodiments of the present disclosure; and

FIG. 11 is a flow diagram using a random forest machine learning model to rank feature importance based on a percentage of retained activity, in accordance with one or more embodiments of the present disclosure;

FIG. 12 is a table showing four features related to monomer type including non-polar, polar, neutral, and cationic (charge) for use in a machine learning model, in accordance with one or more embodiments of the present disclosure;

FIGS. 13A-13C are graphs showing an improvement in the ability of polymers to retain enzymatic activity under thermal stress, in accordance with one or more embodiments of the present disclosure;

FIG. 14 is a table showing ten best performing candidates in stabilizing Chondroitinase ABC, in accordance with one or more embodiments of the present disclosure;

FIG. 15 is a table showing ten worst performing candidates in stabilizing Chondroitinase ABC, in accordance with one or more embodiments of the present disclosure;

FIG. 16 is a table showing ten best performing candidates in stabilizing lipase, in accordance with one or more embodiments of the present disclosure;

FIG. 17 is a table showing ten worst performing candidates in stabilizing lipase, in accordance with one or more embodiments of the present disclosure;

FIG. 18 is a table showing ten best performing candidates in stabilizing Glucose Oxidase, in accordance with one or more embodiments of the present disclosure;

FIG. 19 is a table showing ten worst performing candidates in stabilizing Glucose Oxidase, in accordance with one or more embodiments of the present disclosure;

FIG. 20 is a table showing ten best performing candidates in stabilizing Horseradish peroxidase, in accordance with one or more embodiments of the present disclosure;

FIG. 21 is a table showing ten worst performing candidates in stabilizing Horseradish peroxidase, in accordance with one or more embodiments of the present disclosure;

FIG. 22 is a table with a list of monomers used for the synthesis of heteropolymers, in accordance with one or more embodiments of the present disclosure; and

FIG. 23 is a flowchart of a method for optimizing stable enzyme designs using a fully autonomous controlled/living radical polymerizations (CLRP) flow, in accordance with one or more embodiments of the present disclosure.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising” “including,” and “having” do not limit the scope of a specific claim to the materials or steps recited by the claim.

As used herein, the term “consisting essentially of” limits the scope of a specific claim to the specified materials or steps and those that do not materially affect the basic and novel characteristic or characteristics of the specific claim.

As used herein, terms such as “consisting of” and “composed of” limit the scope of a specific claim to the materials and steps recited by the claim.

All prior patents, publications, and test methods referenced herein are incorporated by reference in their entireties.

Variations, modifications and alterations to embodiments of the present disclosure described above will make themselves apparent to those skilled in the art. All such variations, modifications, alterations and the like are intended to fall within the spirit and scope of the present disclosure, limited solely by the appended claims.

While several embodiments of the present disclosure have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

Any feature or element that is positively identified in this description may also be specifically excluded as a feature or element of an embodiment of the present as defined in the claims.

The disclosure described herein may be practiced in the absence of any element or elements, limitation or limitations, which is not specifically disclosed herein. Thus, for example, in each instance herein, any of the terms “comprising,” “consisting essentially of and “consisting of” may be replaced with either of the other two terms, without altering their respective meanings as defined herein. The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

Embodiments of the present disclosure herein describe systems and method for stabilizing protein designs using polymers. The stabilization of proteins may reduce protein denaturation and prolong enzyme durability, for example. The polymers may provide stabilizing shells on different portions of the protein molecules. The systems and methods disclosed herein leverage the use of machine learning models applied to a plurality of polymers to predict the stabilizing effect of each polymer in the plurality of polymers on a given protein and to generate an experimental flow for measuring and identifying the best polymer compositions and features for optimizing protein stability. The intelligent automation optimizes the protein-polymer interface to enhance protein stability.

Although exemplary embodiments herein are related to the stabilization of enzymes, this is merely for conceptual clarity and not by way of limitation of the embodiments of the present disclosure. The methods and systems taught herein may be applied to stabilizing proteins, biopharmaceuticals, enzymes and the like, using polymers.

In some embodiments, a Design-Build-Test-Learn workflow may be used for identifying quantitative structure-activity relationships (QSARs) that may be used to significantly accelerate the single enzyme nanoparticle (SEN) discovery process. The SEN discovery process disclosed herein implements an intelligent and data-enabled discovery process to optimize the design of stable SENs in harsh conditions. The SEN discovery process may significantly leverage recent advances in high throughput polymer automation to rapidly search through a diverse parameter space in the Design-Build-Test-Learn workflow cycles of experimentation for providing enzyme-specific and robust SEN characteristics that provide the most stable behavior.

In some embodiments, machine learning may be used to help guide the discovery process toward polymer characteristics that provide the most stable behavior. Model data may be continuously validated against a map of the enzyme's assessible surface area (ASA) calculated in the Python Molecular Modelling License (PyMol) molecular visualization system to extract QSARs.

The methods and systems described herein leverage supervised machine learning models, for example, to develop SEN design criteria by elucidating the quantitative structure-activity relationships (QSARs). This may be accomplished by iteratively applying the Design-Build-Test-Learn workflow by implementing a robust and intelligent high throughput process as described hereinbelow. This workflow may utilize a diverse range of polymer characteristics with a machine learning model to rank variable dependencies so as to reveal structure-function relationships that may be otherwise be difficult to determine using hit or miss-type rational designs alone.

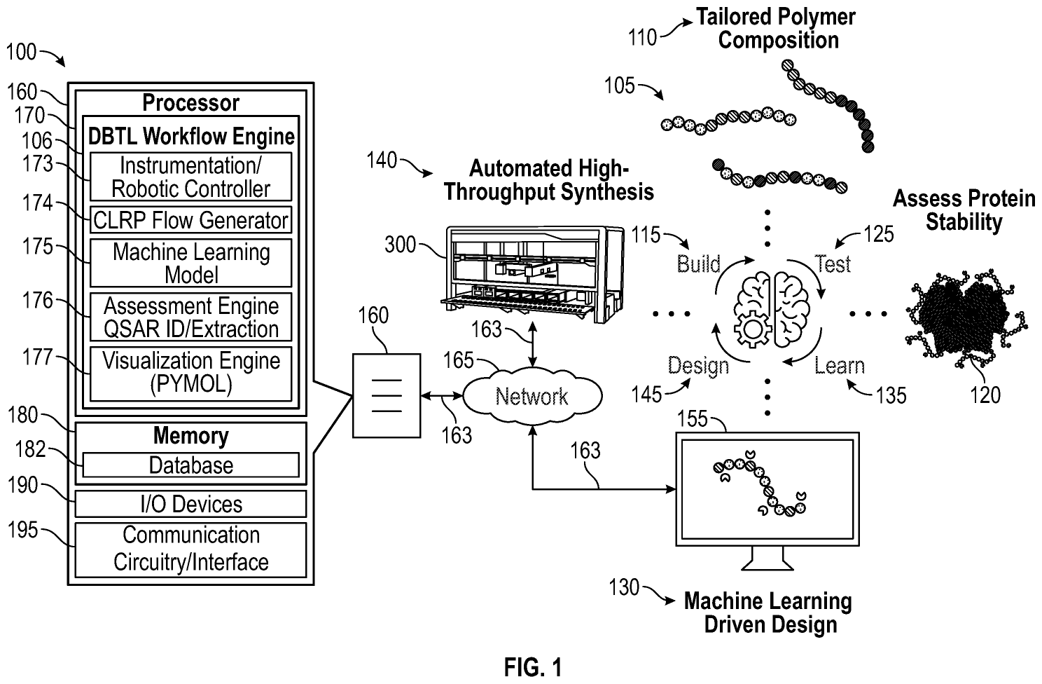

FIG. 1 illustrates a system 100 for implementing a Design-Build-Test-Learn workflow 105 for optimizing stable enzyme designs, in accordance with one or more embodiments of the present disclosure. Design-Build-Test-Learn experimentation workflow 105 may include a design 154 stage, a build 115 stage, a test 125 stage and a learn 135 stage in the experimentation cycle controlled by a computer 160. To optimize the design of single-enzyme nanoparticles (SENs), Design-Build-Test-Learn workflow 105 of experimentation may be used to intelligently sort through polymer characteristics that will provide durable enzyme formulations in harsh conditions. Polymer automation as well as machine learning may be used to sort through this formulation parameter space. Design-Build-Test-Learn (DBTL) workflow 105 shown in FIG. 1 may be used to identify quantitative structure-activity relationships (QSARs) that will significantly accelerate this SEN discovery process.

In some embodiments, in design 145 stage, a desired SEN structure with desired chemical characteristics may be input to a machine learning driven design engine operated by computer 160 for building a fully autonomous controlled/living radical polymerizations (CLRP) flow. Computer 160 may implement the CLRP flow by controlling a fully autonomous instrumentation platform 300 for automated high-throughput synthesis 140 for building a tailored polymer composition 110 with the desired chemical structures and/or chemical characteristics in build 115 stage. In test 125 cycle, computer 160 controlling the CLRP flow may assess protein stability 120 (e.g., enzyme stability) of tailored polymer composition 110. Finally, computer 160 in learn 135 stage may compare the measured results of tailored polymer composition 110 and corresponding protein stability assessment 120 to the originally-designed desired SEN structure with desired chemical characteristics using the machine learning models in design 145 stage. Thus, the polymer automation and machine learning implemented in the Design-Build-Test-Learn (DBTL) cycles of experimentation as shown in FIG. 1 may be used to discover enzyme-specific and robust SENs.

In some embodiments, machine learning models may be used to rank variable dependencies that may reveal structure-function relationships which would otherwise be difficult to determine by rational trial-and-error type classic designs alone. The measured results as compared to the originally-designed desired SEN structure with desired chemical characteristics may be used to update a polymer library database and to subsequently retrain the machine learning models used in a machine learning driven design 130. Model data may be continuously validated against a map of the enzyme's accessible surface area (ASA) calculated in PyMOL to extract QSARs. More detailed characterizations of priority complexes using an ensemble of analytical tools may be used to validate the overall approach.

In some embodiments, computer 160, such as a server, may include a processor 170, a memory 180 storing a database 182, input/output devices 190 such as a display 155 for displaying chemical structure visualizations, and communication circuitry and interface 195 for communicating 163 over a communication network 165 with fully autonomous instrumentation platform 300 and display 155, for example, as shown in FIG. 1. Fully autonomous instrumentation platform 300 may include any suitable instrumentation and/or robotic-based handlers, reagent dispensers, and the like for the automated implementation of any or steps of DBTL workflow 105. Any or all of the instrumentation and/or robotic based handlers may be at the same location and/or in different locations. Computer 160 may be at any location and communicate 163, for example, with any component of fully autonomous instrumentation platform 300 over communication network 165 such as the internet, a network for implementing cloud computing, and/or locally in a laboratory or chemical production facility, for example.

In some embodiments, system 100 as shown in FIG. 1, is merely for conceptual clarity and not by way of limitation of the embodiments disclosed herein. Any, some of the steps, or all of the steps may be fully automated and controller by computer 160. Any, some of the steps, or all of the steps for implementing DBTL workflow 105 for optimizing enzyme designs may be manual and/or automated.

In some embodiments, processor 170 may be configured to execute computer code stored in memory 180 which may cause processor 170 to control any, some, or all the processes described herein. For example, processor 170 may execute a Design-Build-Test-Learn (DBTL) workflow engine 106. DBTL workflow engine 106 may include instrumentation and robotic controller software 173 (e.g., controller script) for controlling any, some or all elements of fully autonomous instrumentation platform 300. DBTL workflow engine 106 may include controlled/living radical polymerizations (CLRP) flow generator software module 174, a machine learning model module 175, an assessment engine/QSAR identification and extraction module 176 for analyzing any or all of the experimental data, and/or a visualization engine 177 such as PyMOL for controlling display 155 so as to output visualizations of chemical structures.

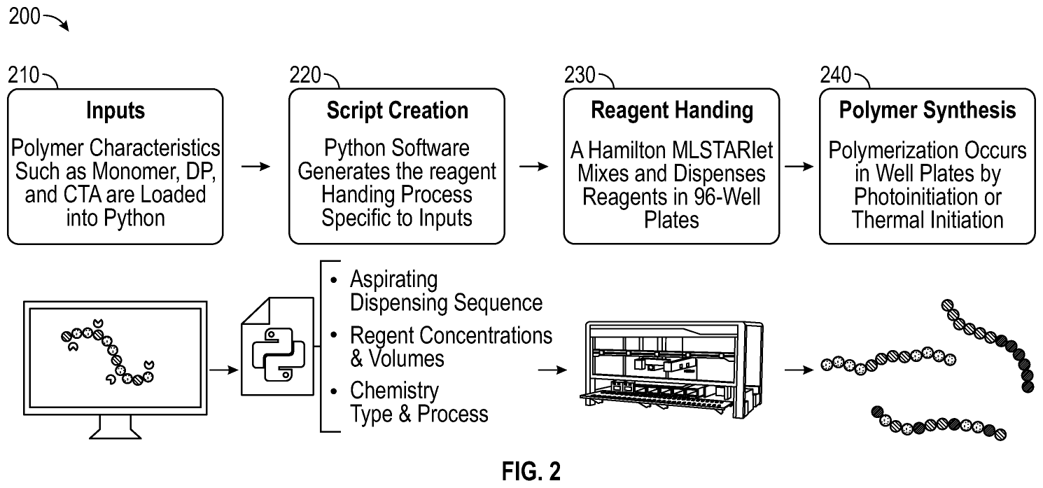

FIG. 2 illustrates a flow diagram of a fully autonomous controlled/living radical polymerizations (CLRP) flow 200 for optimizing stable enzyme designs, in accordance with one or more embodiments of the present disclosure. CLRP flow 200 may include data inputs 210 to DBTL workflow engine 106. CLRP workflow generator 174 may use a database including at least one polymer library to generate script creation 220, such as a Python script generator, for example. Reagent handling 230 and polymer synthesis 240 may be implemented by fully autonomous instrumentation platform 300, controlled by instrumentation and robotic controller software 173 using the generated Python scripts (e.g., controller scripts).

In some embodiments, the reactions described herein may be oxygen tolerant polymerization reactions in that well-defined polymers may be synthesized outside of a fume hood in open well plates (open air environment), for example. The ability to synthesize well-defined polymers in well plates may enable new advances in polymer automation using liquid handling (reagent) robotics. In some embodiments, fully autonomous instrumentation platform 300 may include a Hamilton Microlab STARlet. This instrument is compatible with the fully autonomous CLRP automation as described herein. Using this approach, the synergy between highly customizable liquid handling robotics and oxygen tolerant CLRP to automate advanced polymer synthesis for high throughput and combinatorial polymer research may be implemented.

In some embodiments, data inputs 210 may include polymer characteristics (e.g., input features) such as monomer, the degree of polymerization (DP) and/or a chain transfer agent (CTA) which may be loaded into Python. Script creation generation 220 may use the at least one polymer library stored in database 182. The synthesis processes may be developed using Python, and script creation generation 220 may be used to automate reagent handling, dispensing sequences, and synthesis steps required to create homopolymers, random heteropolymers, and block copolymers in an array of well plates, such as a planar array of 96 well plates, for example, as well as post-polymerization modifications.

Stated differently, script creation generation 220 may generate a mapping of each well plate in the well plate array that may determine the reagents to be dispensed into each well plate in the array of well plates in reagent handling 230 as well as the proteins or enzymes to be stabilized. This mapping may include, for example, the reagent type, reagent concentrations and/or volumes, a number of reagents to dispense into each well plate, aspirating/dispensing sequences, (e.g., the timing and/or sequences of when to dispense the reagents), chemistry type and process, heating steps and/or light activation steps of the mixtures in each of the well plates in the well plate array, and/or any suitable steps needed for (CLRP) flow 200 for polymer synthesis. The term reagent as used herein may include but are not limited to monomers used in the polymerization process, but may include any solutions during the entire experimental flow dispensed into the well plates at any suitable time and/or sequence. The term sample may refer to the reagents and protein dispensed into a single well plate in which polymers are synthesized for stabilizing the protein in accordance with the experimental flow.

In some embodiments, polymer synthesis 240 may occur by photoinitiation and/or by thermal initiation where light and/or heat to any separately, or all of the well plates, in the well plate array. The polymers used to stabilize the proteins may be formed from, but not limited to a polymerization of monomer reagents introduced into the well plates and which are then polymerized.

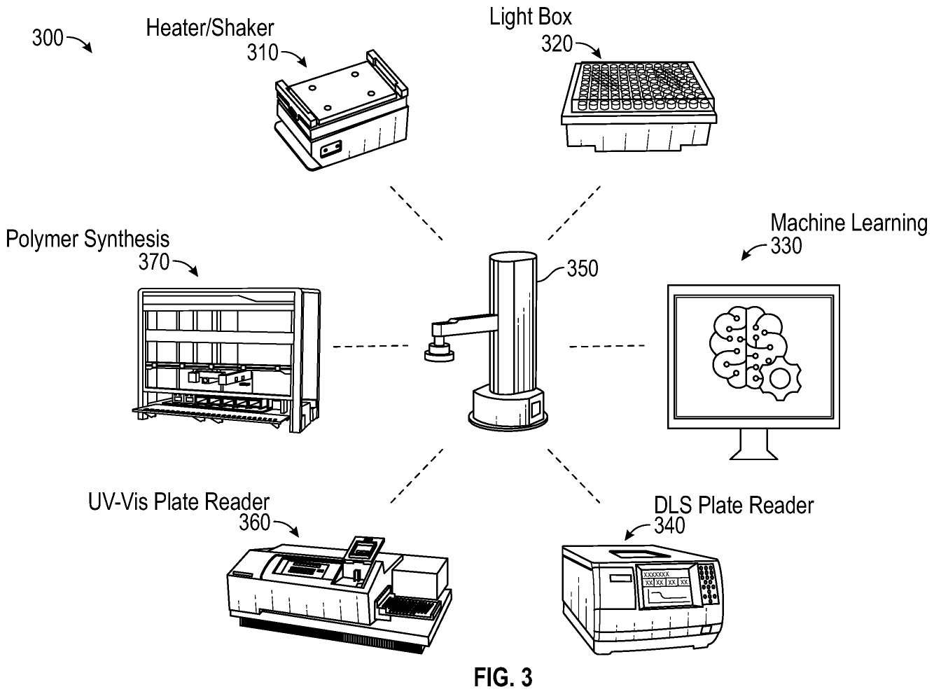

FIG. 3 illustrates fully autonomous instrumentation platform 300 for optimizing stable enzyme designs, in accordance with one or more embodiments of the present disclosure. Fully autonomous instrumentation platform 300 may include, but is not limited to a robotic handler 350, such as a Hudson Robotics PlateCrane EX controlled by instrumentation/robotic controller software 173 for transferring (e.g., moving) well plates between: a Polymer synthesis liquid handler 370, a heater/shaker 310 for the well plate array for applying heat for thermal initiation to the any or all of the well plates in the well plate arrays, a light box 320 for applying light for photoinitiation to the any or all of the well plates in the well plate arrays, such as a custom made Arduino-powered lightbox for photopolymerization, a UV-VIS plate reader 360 such as a SpectraMax UV-Vis plate reader, and a dynamic light scattering (DLS) plate reader. Machine learning model 330 may automatically search for QSARs.

In some embodiments, DBTL Workflow engine 106 may include, but is not limited to LabView software, for example, as the master platform, but any suitable software package may be used. DBTL Workflow engine 106 may further include instrument configuration drivers (e.g., instrumentation/robotic controller module 173) and communication protocols to communicate 163 with the elements of system 100 through communication circuitry and interface 195. All experiments in fully autonomous instrumentation platform 300 may be designed in Labview to direct each instrument to carry out specific functions in CLRP flow 200 including polymer reagent preparation, photoinitiation, tracking of polymerization reaction by fluorescence, polymer dilution into buffer, addition of enzyme, enzyme denaturation by heat or addition of denaturants (i.e. solvents, surfactants, etc.), addition of enzyme substrate, and analysis of enzyme activity by UV-Vis. Machine learning may be used to analyze the data to automatically search and identify QSARs.

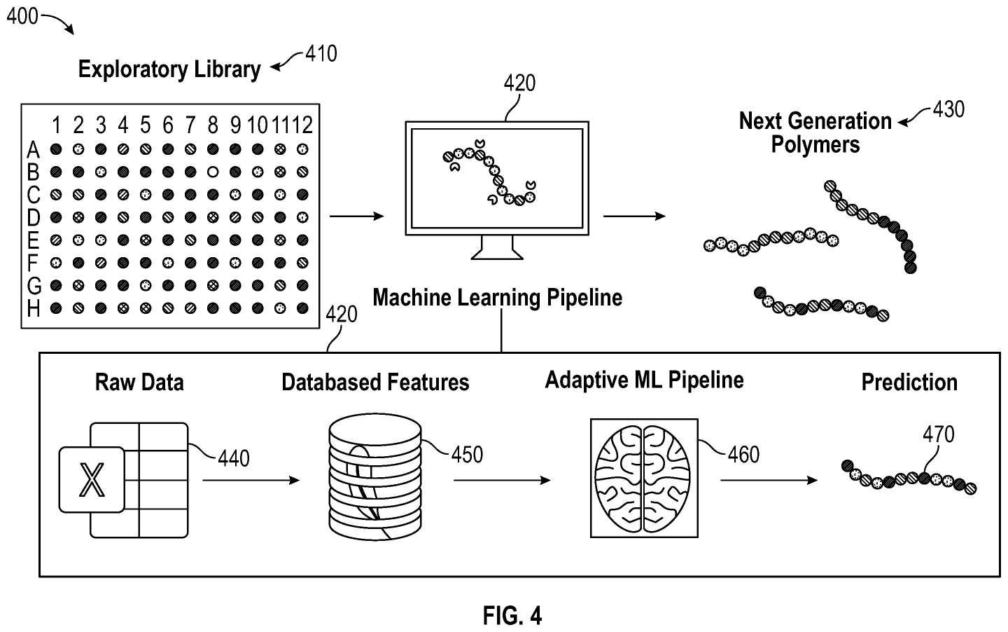

FIG. 4 illustrates a flow diagram of a machine learning guided flow 400 for optimizing stable enzyme designs, in accordance with one or more embodiments of the present disclosure. Machine learning guided flow 400 may include processor 170 fetching an exploratory polymer library 410 stored in database 182 for use in a machine learning pipeline 420 which may yield predictions for next generation polymers 430 based on the experimental raw data obtained from fully autonomous instrumentation platform 300. Machine learning pipeline 420 may include inputting raw data 440 and databased features 450 stored in database 182 to an adaptive machine learning pipeline 460 utilizing a random forest machine learning model so as to output a prediction 470 of polymer feature importance (e.g., a predicted output feature from the machine learning model). Input features of importance and output feature of importance as used herein may respectively refer to chemical structures, and predicted chemical functional characteristics such as % retained activity, for example, for next generation polymers 430. The output features of importance from the machine learning model may be compared to the measured output parameters of interest from CLRP flow 200 for polymer synthesis.

It should be noted that the terms “input feature” and “input feature of importance” may be used interchangeably herein. At least one machine learning model as presented herein uses at least one input feature of importance as an input. The output of the at least one machine learning model may be at least one output feature of importance. The terms “output feature of importance”, “predicted feature of importance”, “predicted feature”, “predicted output feature of importance”, or “output feature” may all be used interchangeably herein.

In some embodiments, to test CLRP flow 200 for polymer synthesis, lipase, a widely used commercial esterase enzyme, may be used to catalyze the hydrolysis of fats in a well plate assay. Since lipase may be often used in harsh conditions such as high temperature and the presence of detergents, thermostable variants from extremophiles have been extensively studied and commercialized.

In some embodiments, 504 different complexes of random heteropolymers (RHPs), for example, may be synthesized using fully autonomous instrumentation platform 300 (Generation 1, G1), which may be diluted, combined with lipase, heated to 80° C. for one hour, and evaluated for % retained enzyme activity. Since the denaturation temperature is 65° C., unstabilized lipase may exhibit a loss in enzyme activity under these conditions.

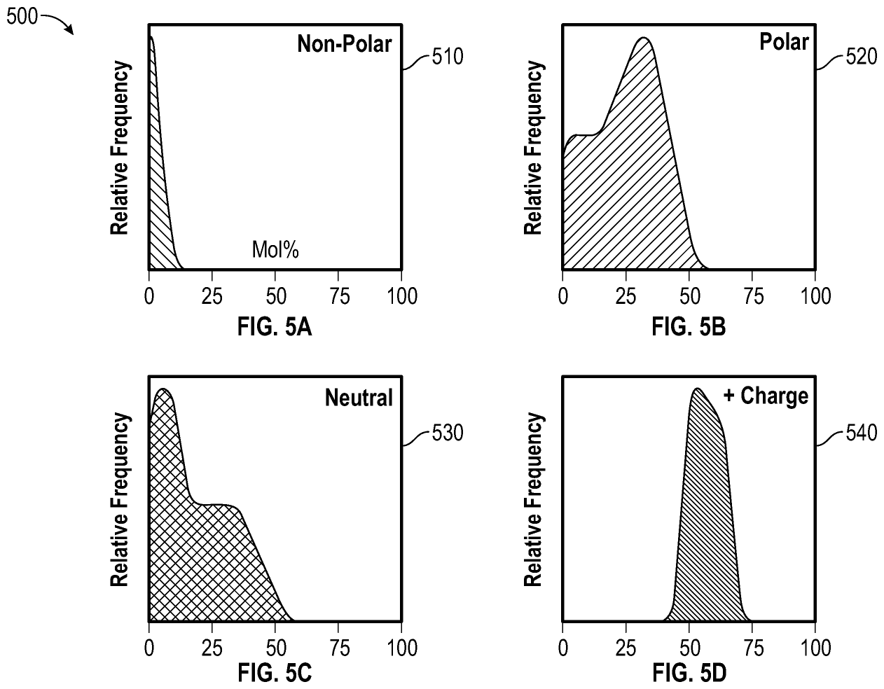

FIGS. 5A-5D are machine learning model-generated plots 500 illustrating feature importance of four tested monomers, in accordance with one or more embodiments of the present disclosure. To generate these plots, a machine learning random forest model was used. Plots of (input) features importance for the four monomers tested (non-polar 510, polar 520, neutral 530, cationic (+charge) 540) established clear trends. The machine learning model may predict that non-polar and neutral monomers have the least contribution to stabilized behavior. Meanwhile, polymers based on polar 520 and cationic 540 monomers may be important features for greater enzyme protection. Therefore, these model outputs may provide a roadmap for Generation 2 (G2) designs (e.g., next generation polymers 430). Examples of non-polar 510 monomers, polar 520 monomers, neutral 530 monomers, cationic (+charge) 540 monomers may respectively include MMA (methyl methacrylate), HPMA (N-(2-hydroxypropyl) methacrylamide), PEGMA (polyethylene glycol methacrylate) and PTMAEMA (poly 2(dimethylamino) ethyl methacrylate)

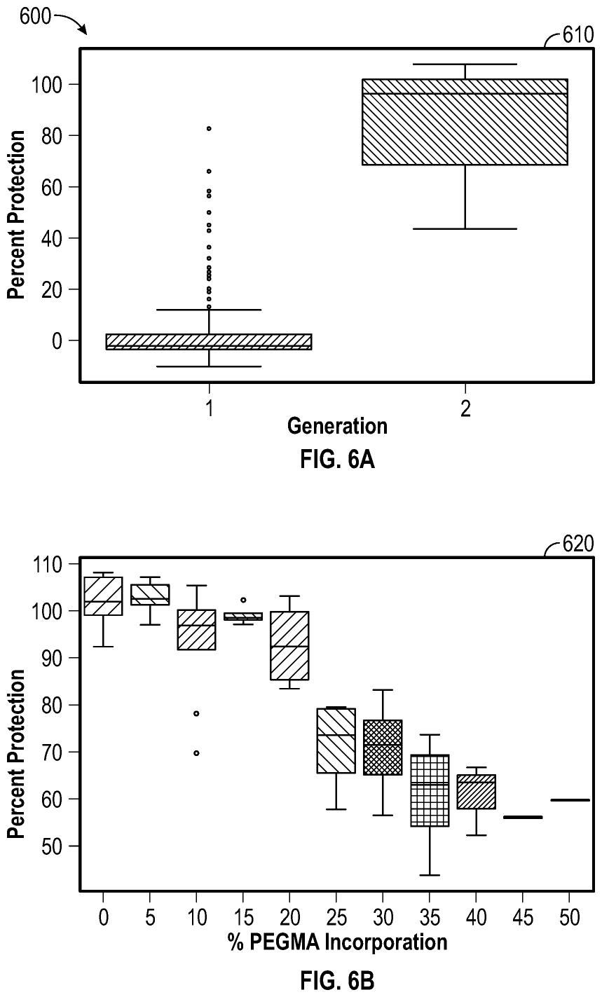

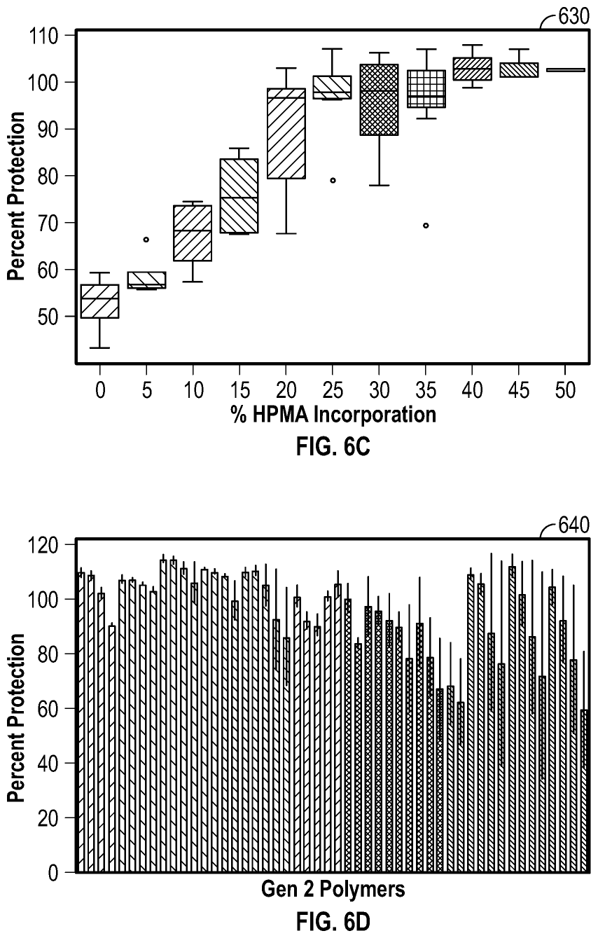

FIGS. 6A-6D are graphs 600 comparing a performance between Generation 1 (G1) versus Generation 2 (G2) polymer libraries and model analysis, in accordance with one or more embodiments of the present disclosure. While the polymers in generation 1 (G1, 504 polymers) polymer library protected 10% of enzyme function, the polymers in optimized generation 2 (G2, 50 polymers) polymer library managed to protect >90% enzyme activity (A) as shown in graph 610. Model analysis shown in graphs 620 and 630 from G2 data indicates clear trends for the neutral (B) and polar (C) monomers which was not detected in G1 due to insufficient data within this new parameter space. Finally, as shown on graph 640, the G2 library was independently resynthesized to assess repeatability of these results (D).

More specifically with regard to graphs 600, the first generation (Generation 1) of 504 polymers (G1) retained greater than 40% enzyme activity, while most provided little or no protection as shown in graph 610. Therefore, based on feature importance, 50 new polymers (G2) were synthesized. In this new generation library (Generation 2 in graph 610), all polymers retained >50% activity, while most retained >90% activity.

The results from the new G2 generation polymers may further reveal new trends which show the influence of the neutral (graph 620) and polar (graph 630) monomers once the polar and cationic monomers have been fine tuned. These G2 polymers were resynthesized in graph 640 in order to confirm high study reproducibility, which may be due to automated CLRP flow 200 for polymer synthesis.

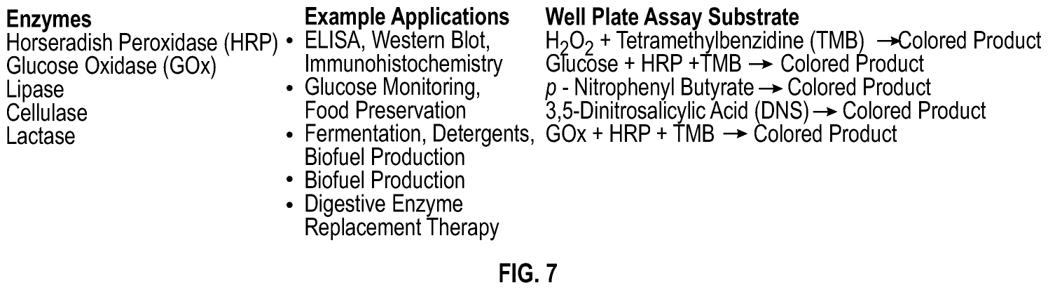

FIG. 7 is a table of five representative enzymes may also be used in an automated controlled/living radical polymerizations (CLRP) flow 200 for polymer synthesis, in accordance with one or more embodiments of the present disclosure. These may include HRP, GOx, lipase, cellulase, and lactase. These enzymes may be used due to their wide applicability to a large number of commercial, industrial, and pharmaceutical applications. Furthermore, these enzymes have convenient well plate format assays that may be easily prepared and may be read on UV-VIS plate reader 360.

In some embodiments, Design-Build-Test-Learn (DBTL) workflow 105 may be validated for representative enzymes, such as the five representative enzymes of the table in FIG. 7. For each enzyme, a QSAR machine learning model may be developed and may be validated with each new dataset. These models may be used in subsequent new designs to create an iterative workflow (e.g., DBTL workflow 105 of FIG. 1) once the accuracy of each enzyme-specific model reaches or exceeds a threshold accuracy.

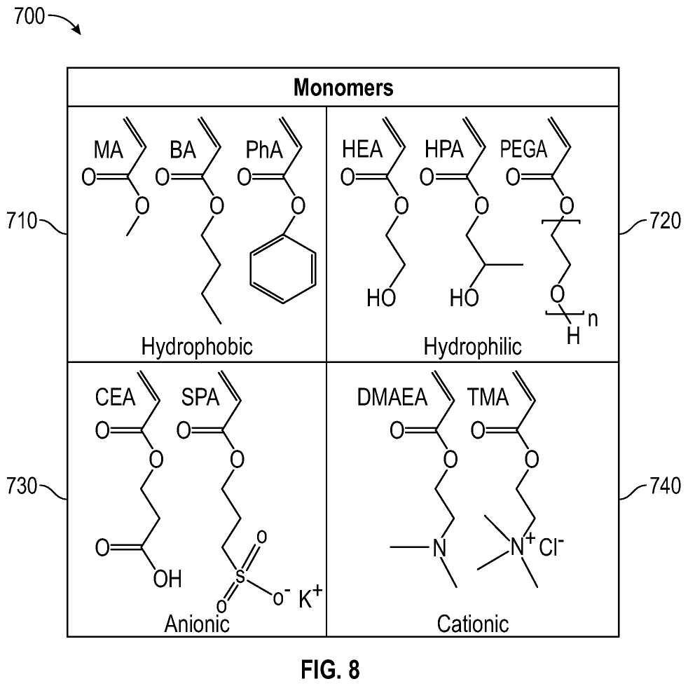

FIG. 8 shows acrylate monomers in a G1 polymer library database, in accordance with one or more embodiments of the present disclosure. For example, a list of 300 polymers may include a variety of homopolymers and random copolymers of varied molecular weight (with degrees of polymerization (DP) of 20-320) so as to attain a maximum diversity in G1. As shown in FIG. 8, hydrophobic monomers 710 may include MA=methyl acrylate, BA=N-butyl acrylate, and PhA=phenyl acrylate. Hydrophilic monomers 720 may include HEA=N-hydroxyethyl acrylate, HPA=N-hydroxyethyl acrylate, and PEGA=poly(ethylene glycol) acrylate. Anionic monomers 730 may include CEA=beta-carboxyethyl acrylate, and SPA=3-sulfopropyl acrylate. Cationic monomers 740 may include DMAEA=2-(N,N-dimethylamino)ethyl acrylate and TMA=2-acryloxyethyltrimethylammonium chloride.

In some embodiments with reference to FIG. 8 and design 145 cycle as shown in FIG. 1, For each enzyme, the Design-Build-Test-Learn (DBTL) workflow 105 may start with an established Generation 1 (G1) library of diverse polymers, such as 504 polymers that may have been already synthesized and inventoried in the laboratory. This polymer library may be designed to have the greatest possible breadth of features so that all possible characteristics may be used to initially train the machine learning model. These characteristics may include a wide molecular weight range (degrees of polymerizations (DP)=20, 40, 80, 160, and 320) and high compositional diversity. A list of 3× hydrophobic, 3× hydrophilic, 2× anionic, and 2× cationic monomers is shown in FIG. 8. This G1 library may serve as an appropriate starting point for experimentation. However, with each new cycle of DBTL workflow 105, new libraries may be synthesized depending on the outputted results from the machine learning model.

In some embodiments, with reference to build 115 cycle in DBTL workflow 105, the automated polymer synthesis process may combine reagents for unique polymers in a well plate array, such as 96 unique polymers in 96 well plates, for example, in a time frame of less than 30 minutes.

In some embodiments, once the reagents are combined, robotic handler 350 may be instructed to transfer the well plate array onto lightbox 320 for photoinitiation. In other embodiments, the automation workflow may accommodate multiple lightboxes for highly multiplexed polymer synthesis. One advantage of using oxygen tolerant photoinduced electron/energy transfer-reversible addition-fragmentation chain-transfer (PET-RAFT) polymerization is that reaction progression may be easily monitored by fluorescence.

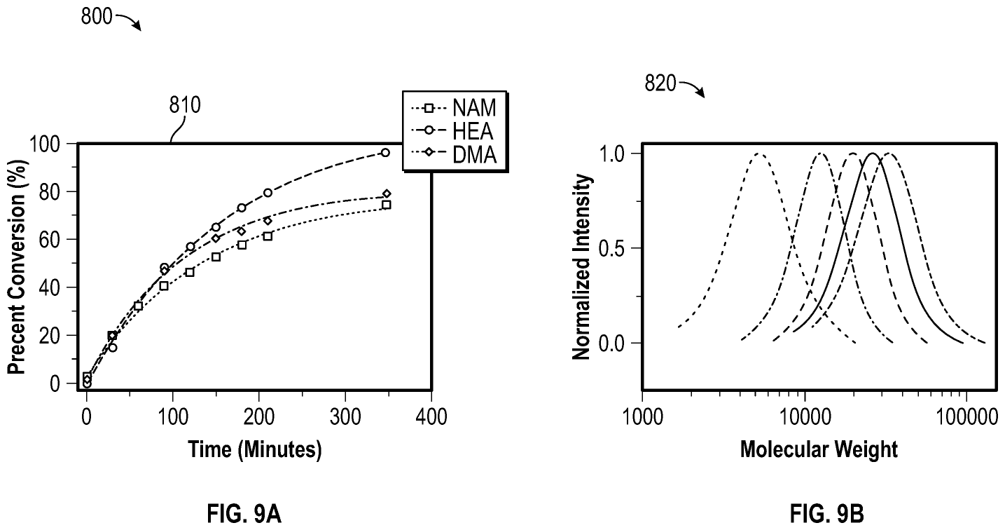

FIGS. 9A-9B are graphs 800 of automated polymer synthesis conversion and a respective molecular weight distribution, in accordance with one or more embodiments of the present disclosure. Robotic handler 350 may transfer the well plate array to UV-VIS plate reader 360 for the online monitoring of conversion as shown in graph 810. Once all polymers have achieved >80% conversion, instrumentation/robotic controller 173 may automatically turned off lightbox 320 to prevent overexposing the reactions to light, which may result in an undesired broadening of the molecular weight distribution.

In some embodiments, turning off lightbox 320 may automatically trigger an automated preparation of analytical plates for high throughput gel permeation chromatography (GPC) as shown in graph 820 in FIG. 9B. All information about polymer-specific reaction kinetics and molecular weights may be saved with sample information in database 182 for experimental tracking.

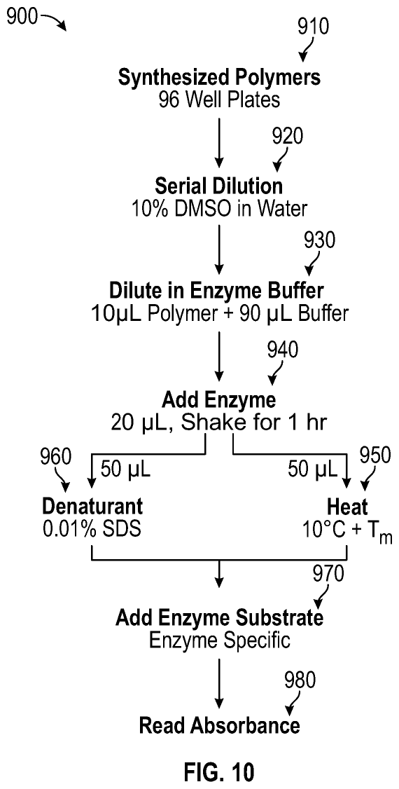

FIG. 10 is a flowchart 900 of a method for measuring protective effects of polymers and enzyme denaturation, in accordance with one or more embodiments of the present disclosure. With reference to test 125 cycle in DBTL workflow 105, test 125 cycle may use heat and denaturants to challenge the protective effects of polymers from enzyme denaturation. The harsh conditions as shown in flowchart 900 were chosen since they may contribute to lost enzyme activity in industrial/commercial processes.

In some embodiments, well plates including polymers (step 910) may be transferred back to the liquid handler 370. Serial dilutions (step 920) may be prepared in 10% Dimethyl sulfoxide (DMSO) in a well plate array of 384 well plates, for example (e.g., 8 dilutions per polymer; 2×384 well plates per 96 polymers). Then, in a new set of 384 well plates, 10 μL of polymer in DMSO may be added to 90 μL of enzyme-specific buffer for another 10× dilution in step 930.

This dilution sequence in step 930 may reduce the risk of polymer precipitation in buffer at high concentrations, so as to ensure that final DMSO concentration with enzyme is below 1% for bringing the 2nd lowest concentration of polymer close to the concentration of enzyme. The Hamilton Microlab STARlet liquid handler (e.g., liquid handler 370) may be uniquely programmed by the manufacturer to detect sample precipitation via unusual pressure changes in aspiration and dispensing. These events may be logged for later tracking of potential errors. While polymer precipitation may be common in these types of experiments, error logging may be used detect these results.

In some embodiments, the system user may receive a notification from fully autonomous instrumentation platform 300 with instructions to load enzyme and enzyme substrate from frozen aliquots in a step 940. Once the enzymes from the frozen aliquots are thawed, fully autonomous instrumentation platform 300 may continue by adding 20 μL enzyme, for example, (concentration may be enzyme specific) followed by robotic placement onto shaker 310 for 1 hour. Then, 50 μL of each polymer/enzyme mixture may be transferred to new well plates (e.g., 384 well plates, for example) for heating and addition of denaturants in a step 950 and a step 960.

In some embodiments, the required melting temperature (Tm) and concentration of surfactant to denature each enzyme may be previously determined from early assay optimization experiments. At first, well plates may be heated 10° C. above Tm for one hour to simulate harsh conditions. Harsh conditions may refer to any condition outside of the protein or enzyme's native environment, such as when horseradish peroxidase (HRP) leaves the roots of horseradish, for example. As improved designs are discovered, this temperature may be gradually increased until the best performing polymers may only retain 10% enzyme activity. Similarly, a predefined concentration of sodium dodecyl sulfate (SDS) to denature each enzyme may be previously determined and may be gradually increased as high performing polymers are discovered.

Finally, enzyme specific substrate and other supportive reagents will be added to measure enzyme function (see FIG. 7) in a step 970. As before, exact conditions will be previously determined in prior optimization experiments guided by the literature. Following an incubation period on the heater/shaker to develop the assay, the well plates may then be transferred to the UV-Vis to measure absorbance for spectrophotometric quantification of % retained enzyme activity relative to positive (no polymer, with heat) and negative (no polymer, no heat) controls. All absorbance data and a log of the experiment from the automation may be saved with polymer information in database 182 for data mining and structure-function analysis.

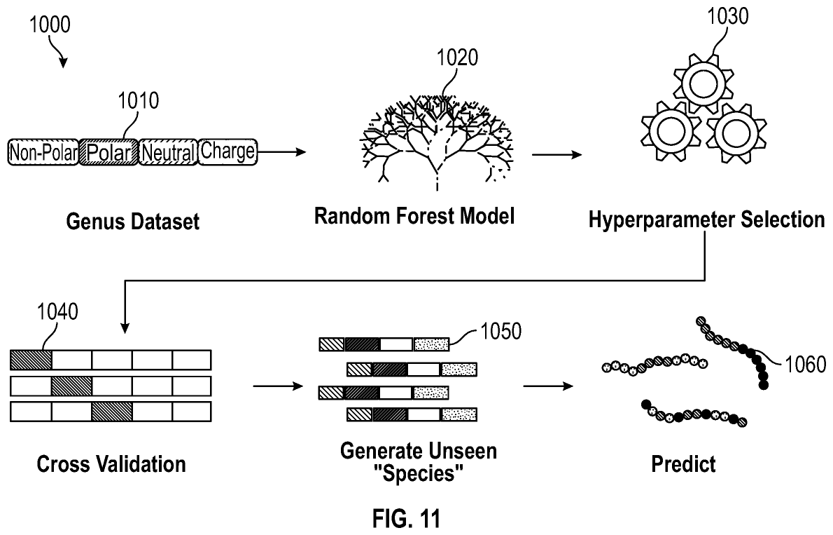

FIG. 11 is a flow diagram 1000 using a random forest machine learning model to rank feature importance based on a percentage of retained activity, in accordance with one or more embodiments of the present disclosure. With reference to learn 135 cycle in DBTL workflow 105, learn 135 cycle may use a random forest model 1020 that may be developed in Python using standard libraries based on a genus dataset 1010 of monomers (e.g., non-polar, polar, neutral and charge monomers).

In some embodiments, data may then be mined and classified by random forest model 1020 to rank feature importance based on % retained activity. This ensemble method of combining many decision trees may be used to robustly classify the feature space while avoiding overfitting. Two additional advantages of this approach may include hyperparameter selection 1030 and cross-validation 1040. By using the hyperparameter traits/characteristics of random forest model 1020 such as tree depth, number of trees, number of samples per leaf, and sample weighting, the model performance may be tuned for accurate fitting. Similarly, cross-validation 1040 automatically splits the data into many different training and testing datasets that may be used to compare model results with the experimental results. This iterative re-training of random forest model 1020 may aid in formulating the best model for the data.

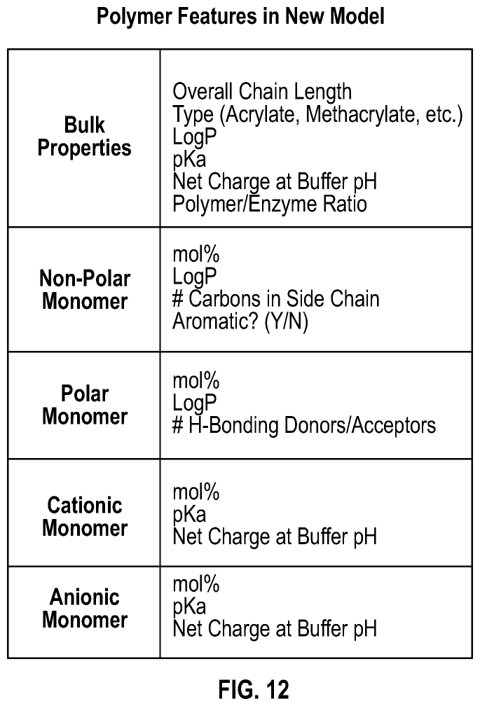

FIG. 12 is a table showing four features related to monomer type including non-polar, polar, neutral, and cationic (charge) for use in a machine learning model, in accordance with one or more embodiments of the present disclosure. In other embodiments, random forest model 1020 may include more input features of importance as listed in the table shown in FIG. 12. These additional features may provide finer characteristics that lead to a more stabilized and/or optimized SENs. Overall chain length as well as polymer type (i.e. acrylates, methacrylates, acrylamides, methacrylamides) may be determined at polymer design. LogP may be automatically calculated once the logP of each monomer is known as well as their mol % in the polymer.

In some embodiments, with each new model generation, random forest model 1020 may to extract at least one output feature importance similar to those seen in FIGS. 5A-5D. Next, the algorithm may be instructed to computationally ‘synthesize’ more than 100,000 possible new polymer designs, for example, within this parameter space into a list. Then, the algorithm may sort through this list and may identify the top 96 new and untested polymer designs that best match the at least one output feature importance. The algorithm may be designed to ensure that a diverse list of new polymer designs (such as 96 polymers, for example) may be selected having with very similar characteristics. Once these new designs are validated, Design-Build-Test-Learn DBTL workflow 105 may be repeated.

In some embodiments, if the data quality provides improved model fit accuracy from the previous experiment cycle, then new results may be included in the improved model to further enhance model accuracy. If median retained enzyme activity exceeds 90% for any new generation, then a new cycle of experimentation may be implemented with increased temperature and denaturant concentration to further challenge SEN behavior (+5° C., +0.5 wt % SDS). This DBTL workflow 105 cycle with increasingly harsh conditions may continue, for example, until new polymer generations with only improved protection by 5% on average with less than 10% retained activity may be achieved. At this point, with the cycle completed having reached an improved protection threshold of 5%, for example, between experimental cycles, a number of the best performing polymers may be assessed such as the five best performing polymers, for example.

In some embodiments, the surface of the five enzymes may be mapped in PyMOL from the protein databank (PDB) (e.g., in database 182). These surface features may be compared to the feature importance map from all machine learning models to establish QSARs. Finally, the best top performing SENs may be further characterized by circular dichroism (CD) spectroscopy, dynamic light scattering (DLS), and isothermal titration calorimetry (ITC).

In some embodiments, with regard to the exemplary lipase enzyme embodiment, processor 170 may use machine learning model 175 (e.g., random forest model) to process a polymer library such as with 500 polymers to determine which monomers for synthesizing any of the 500 polymers in the polymer library are better for Lipase protection (e.g., with a higher % retained activity), which is catalyzing the polymer synthesis. The use of the machine learning model and CLRP flow 200 for polymer synthesis permits checking and optimizing the output features of importance of the synthesized polymers with greater enzyme protection (e.g., highest percentage of retained lipase activity, for example).

In some embodiments, CLRP flow 200 may include screening a polymer library of 500 samples. Each polymer sample input to the machine learning model may include what monomers were used in the polymer, the size of the polymer, what the polymer architecture looks like, etc. model. The machine learning model may map what is the polymer composition is, the polymer size, etc as inputs to an output that is specific to any given experiment goal. So, in the case of lipase activity, the retained activity and/or level of enzyme stability may be the outputs.

In some embodiments, a computational space or chemical landscape may be generated by the machine learning model. The machine learning model may process a large number of possible combinations that can be implemented and verified the robotic system. The measurements may then be used to verify the activity predictions or any suitable scoring metrics. From this, input features of importance may be assessed for each input (e.g., polymer in the library) that yield the predicted stability or activity of the enzyme. Thus, the goal for the case of lipase activity may be to identify the polymer composition yielding high enzyme activity such as 90% that maintains enzyme stability.

In some embodiments, CLRP flow 200 may use robotic handler 350 dispensing of reagents in the well plate array, synthesizing the polymers in the well plate arrays, and analysis by the UV-VIS analytical plate reader. Thus, the inputs (from a user, for example) to the machine learning model may be the polymer and the protein or enzyme to be stabilized including the type of reagents, concentrations, etc, and the polymer synthesis flow as shown in FIG. 11. The output (e.g., the activity and/or level of protein stability) may be obtained from the measured absorbance from the UV-VIS analytical plate reader.

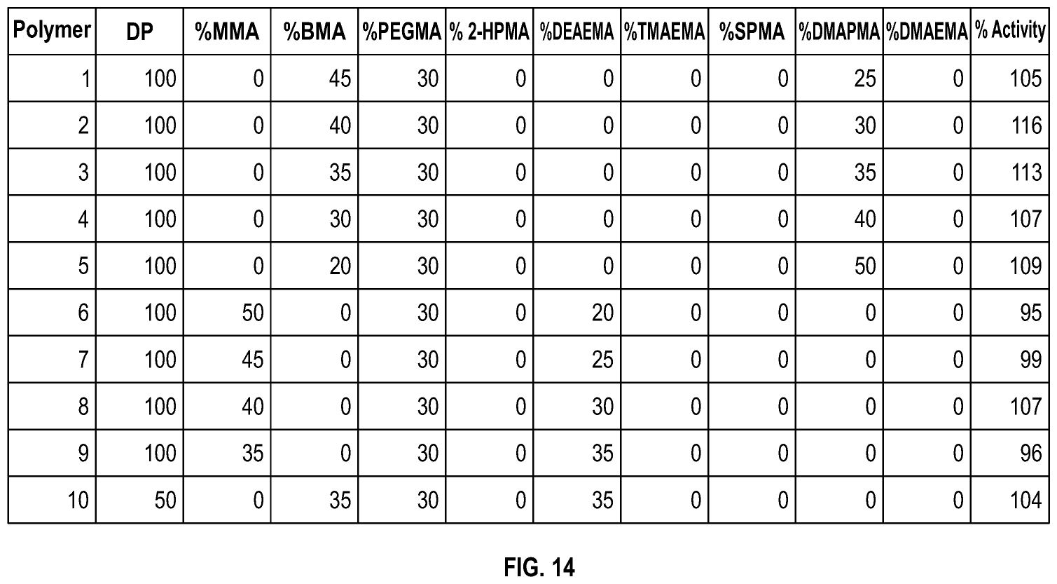

In some embodiments, processor 170 may assign a score to each sample in the well plates in the well plate array based on a comparison between the measured output feature and the desired output feature of importance given by the user. A higher score may be indicative of the higher match between the measured output feature and the desired output feature of importance such as % retained activity and/or protein stability, for example. Processor 170 may identify the well plates having a score higher than a predefined threshold such as the top 10 highest scores, for example. Any suitable threshold may be defined. In other embodiments, the score may be a value directly related to the measured output feature of importance itself such as the % retained activity and/or protein stability, for example, (as later shown in the tables of FIGS. 14, 16, 18, and 20).

In some embodiments, the library of the plurality of polymers stored on 182 may be updated with the experimental results. Machine learning model 175 may be retrained by re-inputting data from the polymer library database (e.g., database 182) in a set of polymers initially identified by machine learning model 175 into machine learning model 175 and matching the predicted output features of importance to the measured output features of importance from the samples (e.g., from the 96 well plates in the well plate array, for example).

In some embodiments, the machine learning pipeline 420 for identifying protein stabilizing polymers may utilize a direct data-driven strategy for discovering novel materials. To accomplish this, the machine learning models 175 may be trained to directly associate input features of importance, such as for example, polymer chemical descriptors (e.g., molecular weight, size, solubility, chemical constituents) with measured output features of importance such as protein stability/activity data acquired by experimentation, for example. Additionally, as feedback driven quantitative structure activity relationship models have been shown to lead to significantly better outcomes than single large batch screens, a heavily reinforcement learning based methodology may be adopted.

In some embodiments, a diverse combinatorial library of 500 chemically distinct polymers, for example, may be used. The effectiveness of these chemically distinct polymers may be assessed for providing stability to the enzymes/protein as described below through established activity assays. In other embodiments, established protein assays may be used that normally render the enzymes inactive through heavy stress (e.g., heat, pH, agitation). The remaining activity of the proteins may be measured in the presence of the polymer library in comparison to the absence of any polymers. Once data is collected, quantitative stability predictions for 100,000 possible polymer permutations, for example, may utilize a random forest regressor model (RF) may be carried out in silico.

In some embodiments, active learning methods may be used to consider both domains of the chemical space that have high and low amounts of information available. This may enable the active learning models to both exploit areas of high information for design and explore areas of low information to maximize learning. After these information thresholds are established, new polymers may be synthesized to both maximize stability (exploitation) and maximize an exploration of a new unknown chemical space (exploration). After synthesis, new generation polymers may be evaluated by previously established protein assays and the collected data may be added to the database 182 for use in further model-based predictions.

In some embodiments, predictions of novel effective protein stabilizers may be determined by using a random forest regressor (RF) machine learning model. To predict highly effective polymer stabilizers, an individually trained RF model may be used for each enzyme independently. The RF model input features (X) (e.g. input features of importance) may include polymer molecular weight, polymer degree of polymerization, and/or relative incorporation of monomer species. Possible monomer species may include, for example, 2-(Diethylamino)ethyl methacrylate, 2-Hydroxypropyl Methacrylate, 2-Sulfopropyl methacrylate, Butyl Methacrylate, 3-(Dimethylamino)propyl methacrylate, Methyl Methacrylate, Poly(ethylene glycol) methyl ether methacrylate, and/or Trimethylammonium chloride ethyl methacrylate for a total of 10 exemplary model input features of importance.

In some embodiments, the RF model output (y) (e.g., output features of importance) may include the corresponding retained protein activity (RPA) for each polymer sample represented as a percentage of the native protein's activity. The models may be trained independently using a randomly selected sample of 80% of the data for training and 20% of the data for validation, for example. During training, RF model hyperparameters (number of trees (100-2000), tree depth (0-10) and number of features (auto or sqrt)) may be optimized through grid searching and 10-fold-cross-validation to minimize the model's mean average error (MAE). After model training, quantitative RPA predictions for 100,000 novel polymer permutations that have not been synthesized and tested may be determined in silico. In addition to predicting RPA, respective prediction variances may be determined by calculating the variance for samples across individual decision trees.

While the data in the following examples has been generated utilizing a RF regressor, this approach may be adapted to other machine learning models, which may provide additional interesting insights. For example, gaussian process regression and recurrent neural networks have been shown to be strong models for active learning and may likely be incorporated in this pipeline.

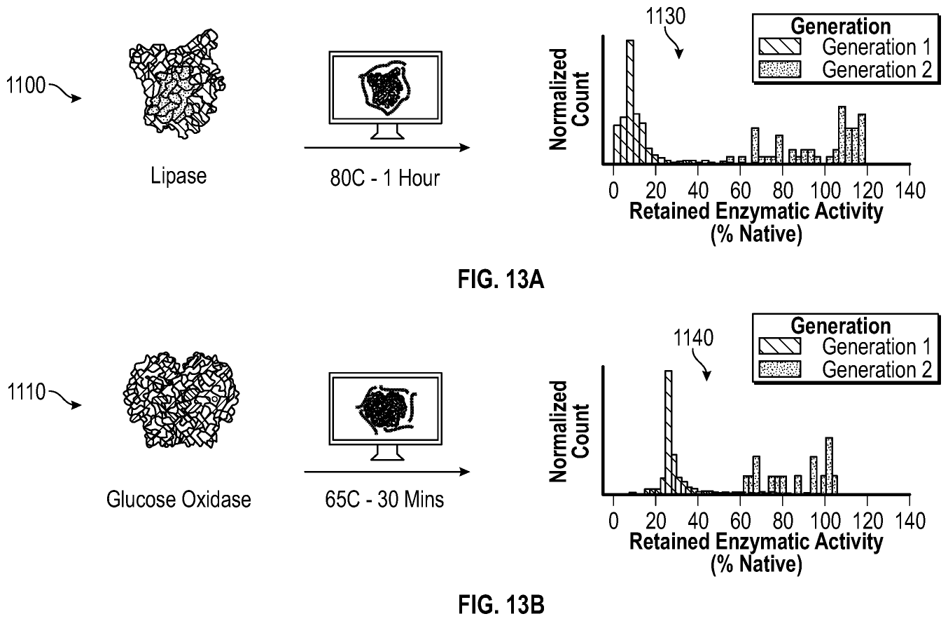

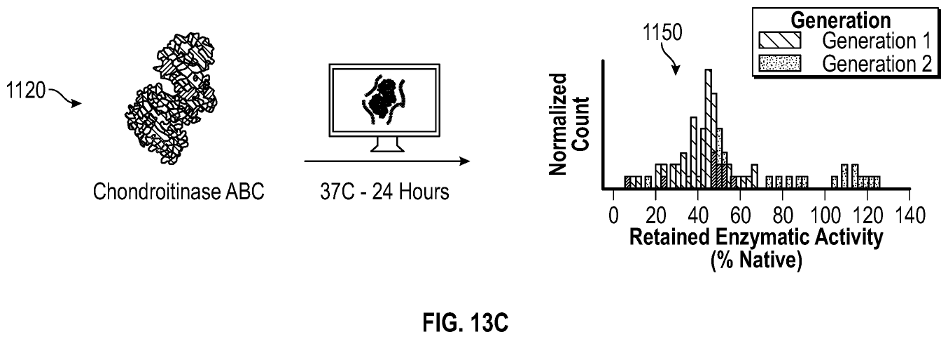

FIGS. 13A-13C are graphs showing an improvement in the ability of polymers to retain enzymatic activity under thermal stress, in accordance with one or more embodiments of the present disclosure. In comparison, second generation polymers designed in silico, outperformed first generation polymers, which make up the combinatorial library.

FIG. 13A shows lipase 1100 protein undergoing a thermal stress of 80 deg C for 1 hour. A graph 1130 illustrates that the retained enzymatic activity of lipase that was synthesized from second generation polymers using the machine learning flow as disclosed herein is significantly higher than lipase synthesized from first generation polymers.

FIG. 13B shows glucose oxidase 1110 protein undergoing a thermal stress of 65 deg C for 30 minutes. A graph 1140 illustrates that the retained enzymatic activity of glucose oxidase that was synthesized from second generation polymers using the machine learning flow as disclosed herein is significantly higher than glucose oxidase synthesized from first generation polymers.

FIG. 13C shows Chondroitinase ABC 1120 protein undergoing a thermal stress of 37 deg C for 24 hours. A graph 1150 illustrates that the retained enzymatic activity of Chondroitinase ABC that was synthesized from second generation polymers using the machine learning flow as disclosed herein is significantly higher than Chondroitinase ABC synthesized from first generation polymers.

In some embodiments, Chondroitinase ABC (chABC), an enzyme derived from Proteus Vulgaris, has shown potential for treating spinal cord injuries because of its ability to degrade certain molecules in scar tissue and promote axonal regeneration. However, it is highly unstable and may lose all of its activity within few hours at 37° C., necessitating the use of repeated injections or multiple infusions for days to weeks during medical treatment for treating the spinal cord injuries (SCI). These infusion systems are highly invasive, infection prone, and clinically problematic to administer.

Therefore, there is a need to explore options that may stabilize this enzyme to increase its therapeutic potential so as to therapeutically reduce the size of SCI scars and facilitate neurons regeneration. Recently, heteropolymers have gained increased attention because of their ability to complex with and preserve enzyme activity in extremely harsh environmental conditions. A laboratory using artificial intelligence and robotics as disclosed herein may be used to identify highly complex polymers that may stabilize chABC at body temperature for long durations. The efficacy and clinical translation potential of top performing polymer-enzyme constructs will be tested in a rodent model for SCI.

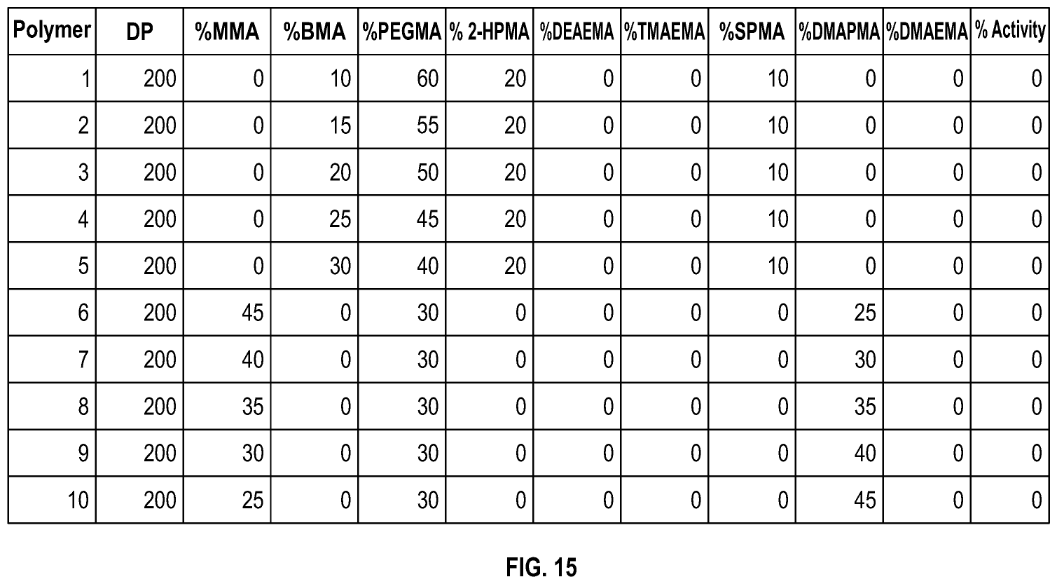

FIG. 14 is a table showing ten best performing candidates in stabilizing Chondroitinase ABC, in accordance with one or more embodiments of the present disclosure. FIG. 15 is a table showing ten worst performing candidates in stabilizing Chondroitinase ABC, in accordance with one or more embodiments of the present disclosure.

It should be noted that in the “polymer” column in each of the following tables is merely a number from 1-10 showing either the 10 best or 10 worst polymers for stabilizing the given enzymes as denoted in the figure caption. They are not related to a specific polymer or enzyme, nor are meant to suggest that the same polymers were used to test a given enzyme for each of the exemplary embodiments shown in FIGS. 14-21 below.

In some embodiments, a few designs were identified that protect chABC and retain its activity for several days as shown in the table of FIG. 14. Polymer samples may be mixed with the enzyme in 96 or 384 well plates, for example, and thermally challenged at 37° C. At the end of the time period that the thermal stress was applied, chondroitin sulfate substrate may be added to calculate the enzymatic activity. The table of FIG. 14 shows the top 10 polymer candidates that retained maximum enzyme activity. candidates that retain >100% chABC activity at end of 24 hours were identified, the table of FIG. 15 shows many polymer-enzyme complexes that did not retain any activity.

Enzymes may play an important role in many industrial and pharmaceutical processes because of their ability to catalyze the reactions at enormous rates that cannot be matched by synthetic counterparts. Lipases are enzymes that may be used as catalysts in place of acid or base catalysts, because of their ability to convert triglycerides as well as free fatty acids (FFAs) to biodiesel. However, lipases are sensitive to surrounding harsh environments such as temperature, low/high pH, or presence of organic solvents.

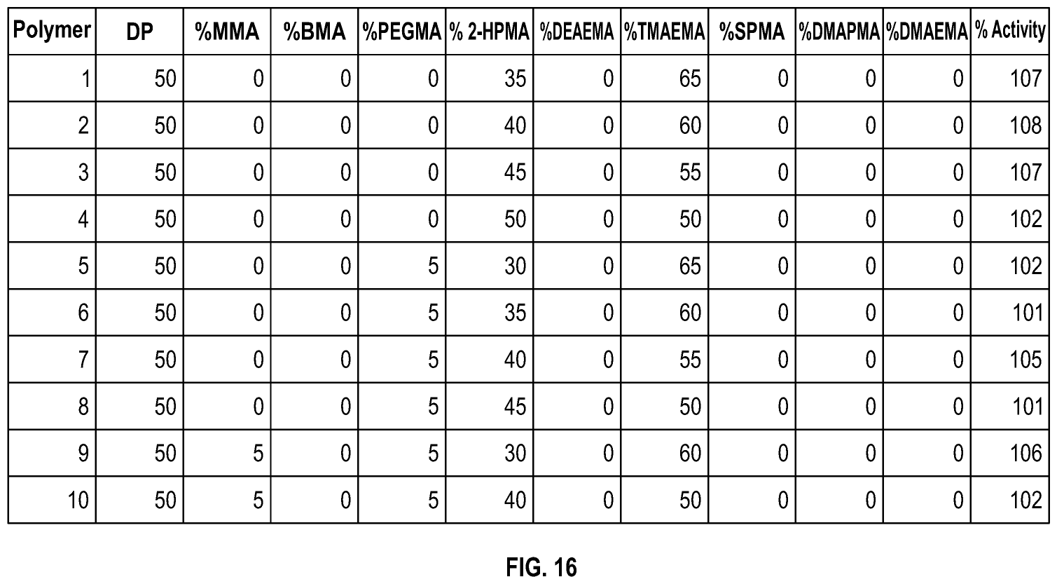

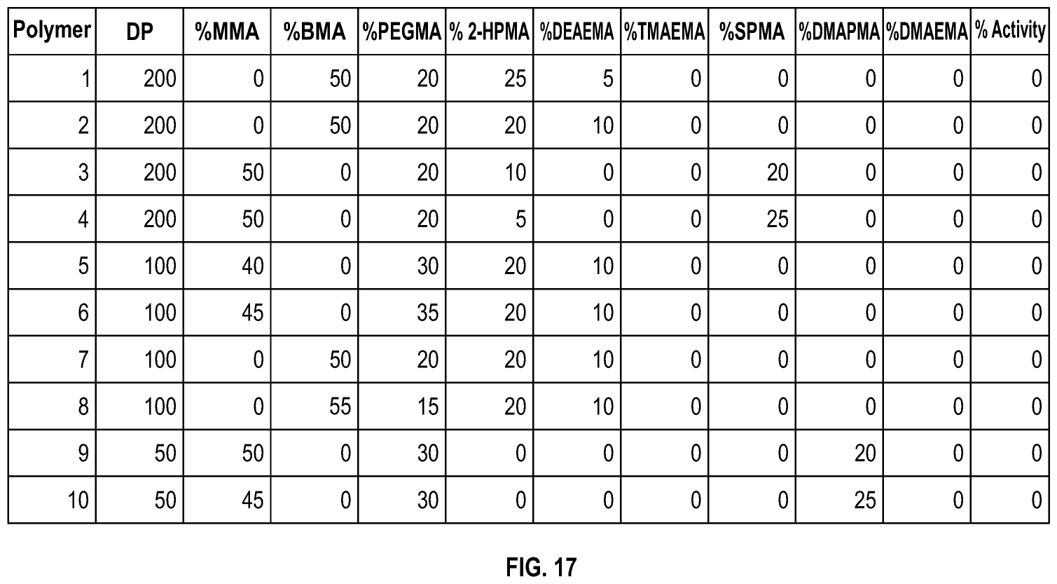

FIG. 16 is a table showing ten best performing candidates in stabilizing lipase, in accordance with one or more embodiments of the present disclosure. FIG. 17 is a table showing ten worst performing candidates in stabilizing lipase, in accordance with one or more embodiments of the present disclosure.

In some embodiments, utilizing the artificial intelligence guided design as disclosed herein, heteropolymers were identified that stabilized Lipase at 80° C. for 1 hr. It is important to note that the native enzyme has a denaturation temperature of 60° C. and loses all activity when heated at that temperature for 30 minutes. The tables shown in FIGS. 16 and 17 respectively demonstrate the best and the worst performing polymer candidates classified by their ability to retain enzymatic activity.

Glucose oxidase, derived from Aspergillus Niger, is an enzyme that oxidizes glucose to gluconolactone and hydrogen peroxide. Naturally produced by some fungi and insects, the main function of GOx is to act as an anti-bacterial and anti-fungal agent by the generation of hydrogen peroxide. This enzyme may be used for various applications like biosensing and food processing. Glucose oxidase may be useful in diverse fields that has necessitated research for improving its stability and increase its catalytic activity under challenging conditions.

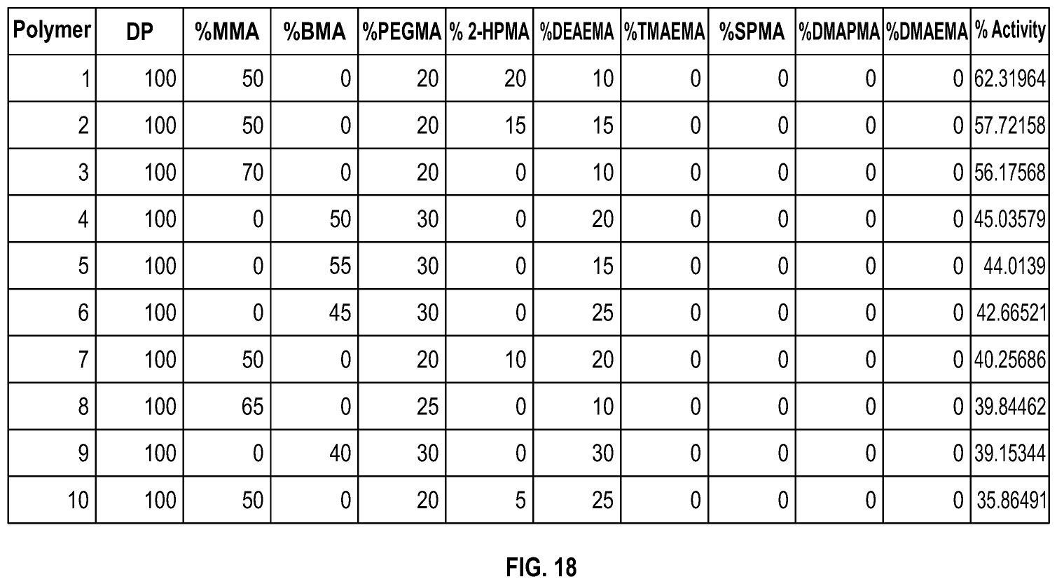

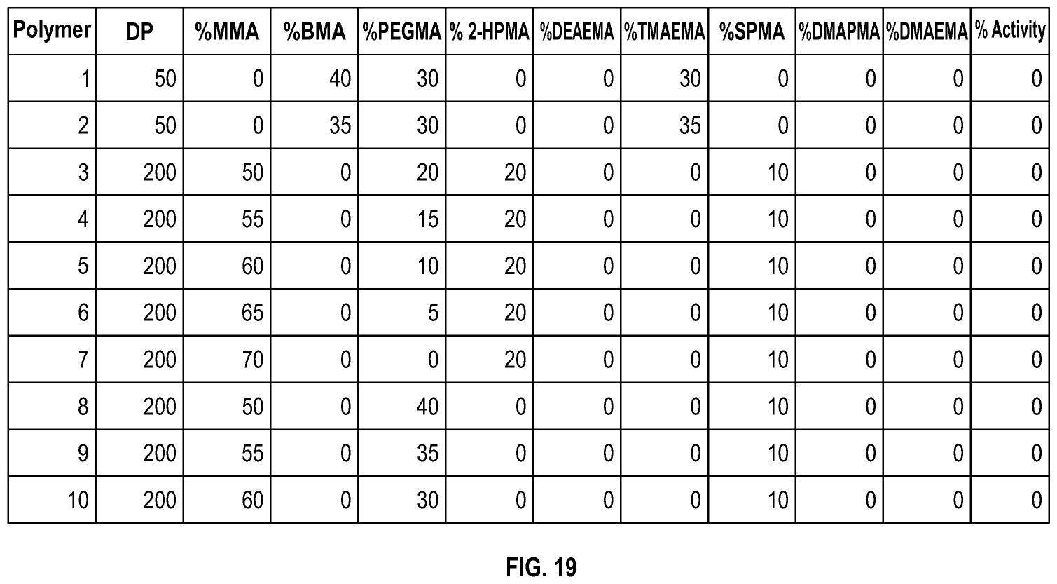

FIG. 18 is a table showing ten best performing candidates in stabilizing Glucose Oxidase, in accordance with one or more embodiments of the present disclosure. FIG. 19 is a table showing ten worst performing candidates in stabilizing Glucose Oxidase, in accordance with one or more embodiments of the present disclosure.

In some embodiments, utilizing the artificial intelligence guided design as disclosed herein, polymers have been identified that retain more than 50% activity when heated at 65° C. for 30 minutes. Native enzyme GOx denatures when heated at 60° C. for 15 minutes. The tables shown in FIGS. 17 and 18 respectively demonstrate the best and worst performing polymers for stabilizing glucose oxidase.

Horseradish peroxidase (HRP) is an important heme group enzyme that catalyzes a wide range of organic substrates in the presence of peroxide. Horseradish peroxidase has may uses in diagnostic and biosensing applications. However, HRP is highly unstable and loses all activity within 5 minutes when heated at its denaturation temperature of 55° C.

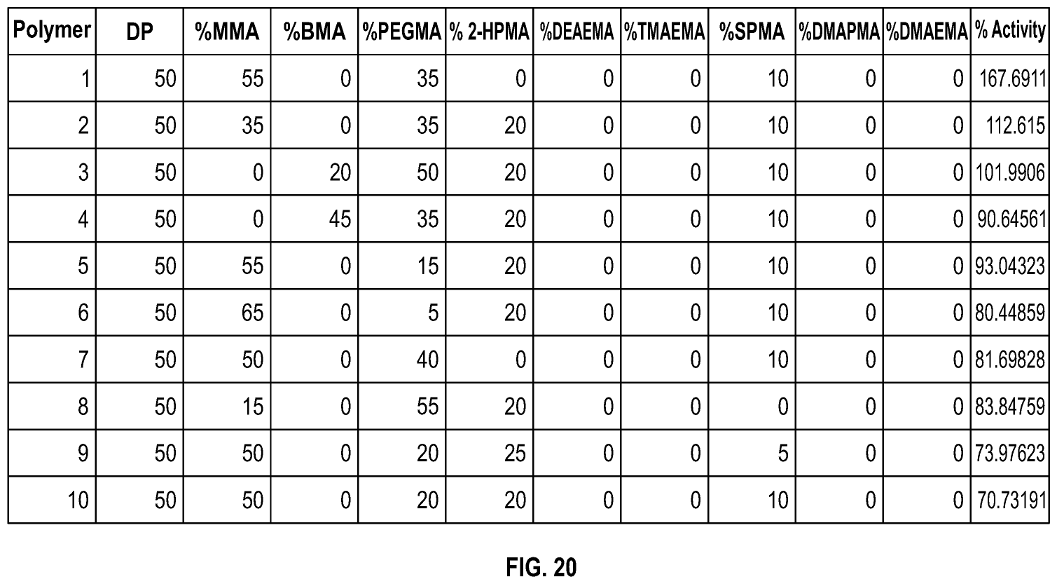

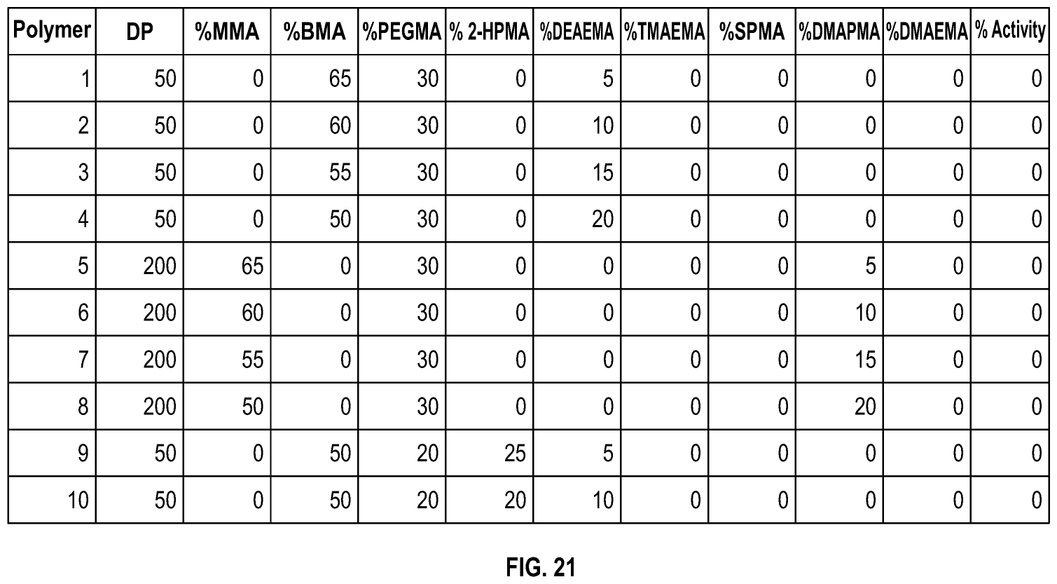

FIG. 20 is a table showing ten best performing candidates in stabilizing Horseradish peroxidase, in accordance with one or more embodiments of the present disclosure. FIG. 21 is a table showing ten worst performing candidates in stabilizing Horseradish peroxidase, in accordance with one or more embodiments of the present disclosure.

In some embodiments, utilizing the artificial intelligence guided design as disclosed herein and a library of heteropolymers, several candidates have been identified that retain more than 100% activity of the native enzyme. The tables shown in FIGS. 20 and 21 respectively demonstrate the best and worst performing polymers for stabilizing HRP.

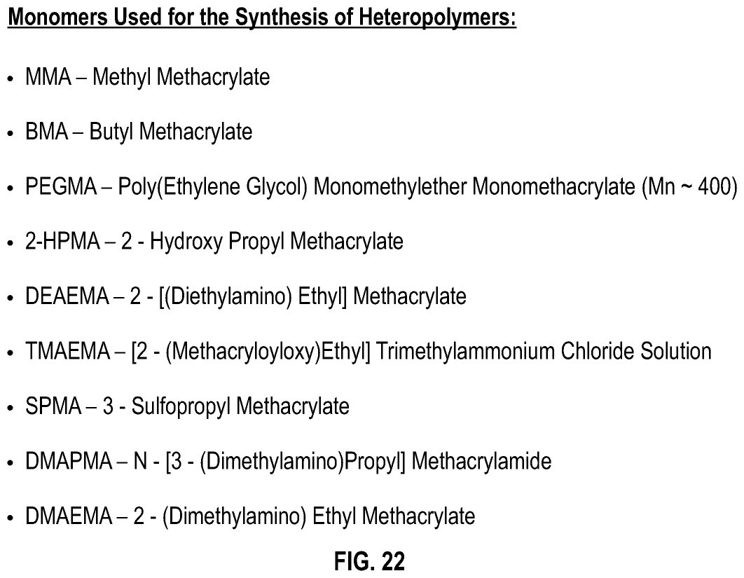

FIG. 22 is a table with a list of monomers used for the synthesis of heteropolymers, in accordance with one or more embodiments of the present disclosure. These are the monomers shown in FIGS. 14-21

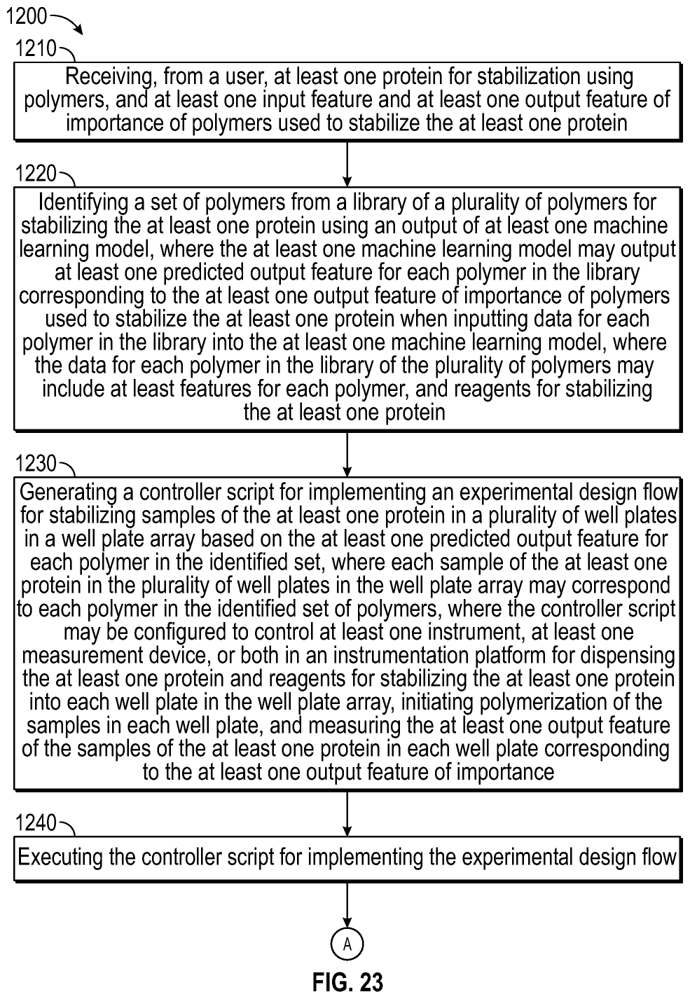

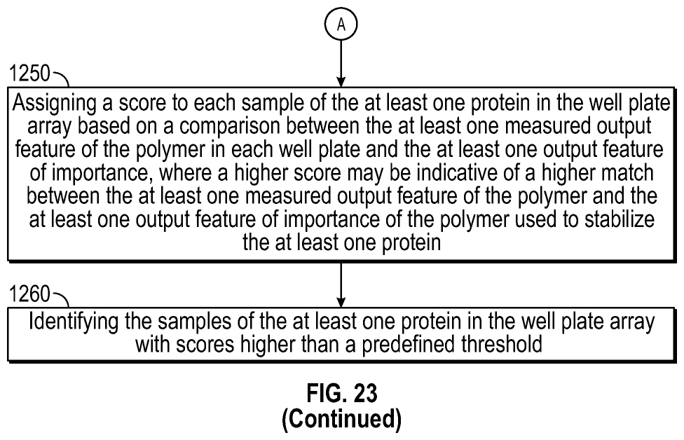

FIG. 23 is a flowchart of a method 1200 for optimizing stable enzyme designs using the fully autonomous controlled/living radical polymerizations (CLRP) flow 200, in accordance with one or more embodiments of the present disclosure. The method 1200 may be performed by the processor 170.

The method 1200 may include receiving 1210, from a user, at least one protein for stabilization using polymers, and at least one input feature and at least one output feature of importance of polymers used to stabilize the at least one protein.

The method 1200 may include identifying 1220 a set of polymers from a library of a plurality of polymers for stabilizing the at least one protein using an output of at least one machine learning model, where the at least one machine learning model may output at least one predicted output feature for each polymer in the library corresponding to the at least one output feature of importance of polymers used to stabilize the at least one protein when inputting data for each polymer in the library into the at least one machine learning model, where the data for each polymer in the library of the plurality of polymers may include at least features for each polymer, and reagents for stabilizing the at least one protein.

The method 1200 may include generating 1230 a controller script for implementing an experimental design flow for stabilizing samples of the at least one protein in a plurality of well plates in a well plate array based on the at least one predicted output feature for each polymer in the identified set, where each sample of the at least one protein in the plurality of well plates in the well plate array may correspond to each polymer in the identified set of polymers, where the controller script may be configured to control at least one instrument, at least one measurement device, or both in an instrumentation platform for dispensing the at least one protein and reagents for stabilizing the at least one protein into each well plate in the well plate array, initiating polymerization of the samples in each well plate, and measuring the at least one output feature of the samples of the at least one protein in each well plate corresponding to the at least one output feature of importance.

The method 1200 may include executing 1240 the controller script for implementing the experimental design flow.

The method 1200 may include assigning 1250 a score to each sample of the at least one protein in the well plate array based on a comparison between the at least one measured output feature of the polymer in each well plate and the at least one output feature of importance, where a higher score may be indicative of a higher match between the at least one measured output feature of the polymer and the at least one output feature of importance of the polymer used to stabilize the at least one protein.

The method 1200 may include identifying 1250 the samples of the at least one protein in the well plate array with scores higher than a predefined threshold.

In some embodiments, exemplary inventive, specially programmed computing systems/platforms with associated devices may be configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less.

In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiment, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a tweet, a map, an entire application (e.g., a calculator), etc.

In some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) AmigaOS, AmigaOS 4, (2) FreeBSD, NetBSD, OpenBSD, (3) Linux, (4) Microsoft Windows, (5) OpenVMS, (6) OS X (Mac OS), (7) OS/2, (8) Solaris, (9) Tru64 UNIX, (10) VM, (11) Android, (12) Bada, (13) BlackBerry OS, (14) Firefox OS, (15) iOS, (16) Embedded Linux, (17) Palm OS, (18) Symbian, (19) Tizen, (20) WebOS, (21) Windows Mobile, (22) Windows Phone, (23) Adobe AIR, (24) Adobe Flash, (25) Adobe Shockwave, (26) Binary Runtime Environment for Wireless (BREW), (27) Cocoa (API), (28) Cocoa Touch, (29) Java Platforms, (30) JavaFX, (31) JavaFX Mobile, (32) Microsoft XNA, (33) Mono, (34) Mozilla Prism, XUL and XULRunner, (35) .NET Framework, (36) Silverlight, (37) Open Web Platform, (38) Oracle Database, (39) Qt, (40) SAP NetWeaver, (41) Smartface, (42) Vexi, and (43) Windows Runtime.

In some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, and/or objects. For example, such projections may be a visual overlay for a mobile augmented reality (MAR) application.

In some embodiments, exemplary inventive computer-based systems of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000, and so on. As used herein, the term “user” shall have a meaning of at least one user.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RCS, CAST and Skipjack), cryptographic hash algorithms (e.g., MD5, RIPEMD-160, RTRO, SHA-1, SHA-2, Tiger (TTH),WHIRLPOOL, RNGs).

The aforementioned examples are, of course, illustrative and not restrictive.

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

As used herein, the term “synthesis, synthesize” or variations of the word shall have the meaning of at least one chemical reaction producing at least chemical product. In some embodiments the term “synthesis” means at least one chemical is made by any process.

A method may include:

receiving, by a processor, from a user, at least one protein for stabilization using polymers, and at least one input feature and at least one output feature of importance of polymers used to stabilize the at least one protein;

identifying, by the processor, a set of polymers from a library of a plurality of polymers for stabilizing the at least one protein using an output of at least one machine learning model;

wherein the at least one machine learning model may output at least one predicted output feature for each polymer in the library corresponding to the at least one output feature of importance of polymers used to stabilize the at least one protein when inputting data for each polymer in the library into the at least one machine learning model;

wherein the data for each polymer in the library of the plurality of polymers may include at least:
(i) features for each polymer, and
(ii) reagents for stabilizing the at least one protein;

generating, by the processor, a controller script for implementing an experimental design flow for stabilizing samples of the at least one protein in a plurality of well plates in a well plate array based on the at least one predicted output feature for each polymer in the identified set;

wherein each sample of the at least one protein in the plurality of well plates in the well plate array may correspond to each polymer in the identified set of polymers;

wherein the controller script may be configured to control at least one instrument, at least one measurement device, or both in an instrumentation platform for:
(i) dispensing the at least one protein and reagents for stabilizing the at least one protein into each well plate in the well plate array;
(ii) initiating polymerization of the samples in each well plate; and
(iii) measuring the at least one output feature of the samples of the at least one protein in each well plate corresponding to the at least one output feature of importance;

executing, by the processor, the controller script for implementing the experimental design flow;

assigning, by the processor, a score to each sample of the at least one protein in the well plate array based on a comparison between the at least one measured output feature of the polymer in each well plate and the at least one output feature of importance;

wherein a higher score may be indicative of a higher match between the at least one measured output feature of the polymer and the at least one output feature of importance of the polymer used to stabilize the at least one protein; and

identifying, by the processor, the samples of the at least one protein in the well plate array with scores higher than a predefined threshold.

In some embodiments, the at least one input feature and the at least one output feature of importance of polymers used to stabilize the at least one protein respectively may include polymer structural features and polymer functional features for stabilizing the at least one protein.

In some embodiments, the at least one output feature of importance may include an activity of the at least one protein.

In some embodiments, the method may further include updating, by the processor, the library with the at least one measured output feature from the samples of the at least one protein corresponding to polymers in the plurality of polymers in the identified set.

In some embodiments, the method may further include retraining, by the processor, the at least one machine learning model by inputting the data for each polymer in the identified set of polymers into the at least one machine learning model and matching the at least one predicted output feature to the at least one measured output feature from the samples of the at least one protein corresponding to polymers in the plurality of polymers in the identified set.

In some embodiments, the at least one machine learning model may be a random forest machine learning model.

In some embodiments, the at least one protein may be an enzyme.

In some embodiments, the enzyme may be selected from the group consisting of; horseradish peroxidase (HRP), glucose oxidase (GOx), Chondroitinase ABC (chABC), lipase, cellulase, and lactase.

In some embodiments, the reagents for stabilizing the enzyme may include four monomers, and the stabilized enzyme may include four parts corresponding to the four monomers.

In some embodiments, a system may include an instrumentation platform including at least one instrument, at least one measurement device, or both, and at least one processor. The at least one processor may be configured to:

receive from a user, at least one protein for stabilization using polymers, and at least one input feature and at least one output feature of importance of polymers used to stabilize the at least one protein;

identify a set of polymers from a library of a plurality of polymers for stabilizing the at least one protein using an output of at least one machine learning model;

wherein the at least one machine learning model may output at least one predicted output feature for each polymer in the library corresponding to the at least one output feature of importance of polymers used to stabilize the at least one protein when inputting data for each polymer in the library into the at least one machine learning model;

wherein the data for each polymer in the library of the plurality of polymers may include at least:
(i) features for each polymer, and
(ii) reagents for stabilizing the at least one protein;

generate a controller script for implementing an experimental design flow for stabilizing samples of the at least one protein in a plurality of well plates in a well plate array based on the at least one predicted output feature for each polymer in the identified set;

wherein each sample of the at least one protein in the plurality of well plates in the well plate array may correspond to each polymer in the identified set of polymers;

wherein the controller script may be configured to control at least one instrument, at least one measurement device, or both in an instrumentation platform for:
(i) dispensing the at least one protein and reagents for stabilizing the at least one protein into each well plate in the well plate array;
(ii) initiating polymerization of the samples in each well plate; and
(iii) measuring the at least one output feature of the samples of the at least one protein in each well plate corresponding to the at least one output feature of importance;

execute the controller script for implementing the experimental design flow;

assign a score to each sample of the at least one protein in the well plate array based on a comparison between the at least one measured output feature of the polymer in each well plate and the at least one output feature of importance;

wherein a higher score may be indicative of a higher match between the at least one measured output feature of the polymer and the at least one output feature of importance of the polymer used to stabilize the at least one protein; and

identify the samples of the at least one protein in the well plate array with scores higher than a predefined threshold.

In some embodiments, the at least one input feature and the at least one output feature of importance of polymers used to stabilize the at least one protein respectively comprise polymer structural features and polymer functional features for stabilizing the at least one protein.

In some embodiments, the at least one output feature of importance may include an activity of the at least one protein.

In some embodiments, the at least one processor may be further configured to update the library with the at least one measured output feature from the samples of the at least one protein corresponding to polymers in the plurality of polymers in the identified set.

In some embodiments, the at least one processor may be further configured to retrain the at least one machine learning model by inputting the data for each polymer in the identified set of polymers into the at least one machine learning model and matching the at least one predicted output feature to the at least one measured output feature from the samples of the at least one protein corresponding to polymers in the plurality of polymers in the identified set.

In some embodiments, the at least one machine learning model may be a random forest machine learning model.

In some embodiments, the at least one protein may be an enzyme.

In some embodiments, the enzyme may be selected from the group consisting of; horseradish peroxidase (HRP), glucose oxidase (GOx), Chondroitinase ABC (chABC), lipase, cellulase, and lactase.

In some embodiments, the reagents for stabilizing the enzyme comprises four monomers, and wherein the stabilized enzyme comprises four parts corresponding to the four monomers.

In some embodiments, monomers used for stabilizing the at least one protein may be selected from the group consisting of Methyl methacrylate (MMA), Butyl methacrylate (BMA), Poly(ethylene glycol) Monomethylether Monomethacrylate (PEGMA), 2-Hydroxypropyl methacrylate (2-HPMA), 2-[(diethylamino) ethyl] methacrylate (DEAEMA), [2-(methacryloyloxy)ethyl]trimethylammonium chloride solution (TMAEMA), 3-Sulfopropyl methacrylate (SPMA), N-[3-(Dimethylamino)propyl]methacrylamide (DMAPMA), and 2-(Dimethylamino)ethyl methacrylate (DMAEMA).

In some embodiments, a composition may include an at least one polymer from a genus of polymers, such as shown in the tables of FIGS. 14, 16, 18, and 20 and referenced in FIG. 22; and an at least one protein from a genus proteins such as shown in the tables of FIGS. 14, 16, 18, and 20, where the composition has a sufficient amount of the at least one polymer to stabilize the at least one protein in an open well plate environment so that the at least one protein has an activity as specified in the tables of FIGS. 14, 16, 18, and 20 in the open well plate environment when is tested by any suitable testing method/standard such as described, for example, in the following references: (1) B. Panganiban, et al, “Random heteropolymers preserve protein function in foreign environments”, Science, 359 (2018) 1239-1243, (2) S.-i. Sawada, et al, “Nano-encapsulation of lipase by self-assembled nanogels: Induction of high enzyme activity and thermal stabilization”, Macromol. Biosci., 10 (2010) 353-358, and (3) A. Raspa, et al, “Feasible stabilization of chondroitinase abc enables reduced astrogliosis in a chronic model of spinal cord injury”, CNS Neurosci. Ther. 25 (2019) 86-100.

Variations, modifications and alterations to embodiments of the present disclosure described above will make themselves apparent to those skilled in the art. All such variations, modifications, alterations and the like are intended to fall within the spirit and scope of the present disclosure, limited solely by the appended claims.

While several embodiments of the present disclosure have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

Any feature or element that is positively identified in this description may also be specifically excluded as a feature or element of an embodiment of the present as defined in the claims.

The disclosure described herein may be practiced in the absence of any element or elements, limitation or limitations, which is not specifically disclosed herein. Thus, for example, in each instance herein, any of the terms “comprising,” “consisting essentially of and “consisting of” may be replaced with either of the other two terms, without altering their respective meanings as defined herein. The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

1-21. (canceled)

22. A method, comprising:
receiving, by a processor, from a user, at least one protein for stabilization using polymers, and at least one input feature and at least one output feature of importance of polymers used to stabilize the at least one protein;
identifying, by the processor, a set of polymers from a library of a plurality of polymers for stabilizing the at least one protein using an output of at least one machine learning model;
wherein the at least one machine learning model outputs at least one predicted output feature for each polymer in the library corresponding to the at least one output feature of importance of polymers used to stabilize the at least one protein when inputting data for each polymer in the library into the at least one machine learning model;
wherein the data for each polymer in the library of the plurality of polymers comprises at least:
(i) features for each polymer, and
(ii) reagents for stabilizing the at least one protein;
generating, by the processor, a controller script for implementing an experimental design flow for stabilizing samples of the at least one protein based on the at least one predicted output feature for each polymer in the identified set;
receiving, by the processor, based on the controller script, a measurement of the at least one output feature of the samples of the at least one protein corresponding to the at least one output feature of importance;
assigning, by the processor, a score to each sample of the at least one protein based on a comparison between the at least one measured output feature of the polymer and the at least one output feature of importance;
wherein a higher score is indicative of a higher match between the at least one measured output feature of the polymer and the at least one output feature of importance of the polymer used to stabilize the at least one protein; and
identifying, by the processor, the samples of the at least one protein with scores higher than a predefined threshold.

23. The method of claim 22, wherein each sample of the at least one protein corresponds to each polymer in the identified set of polymers.

24. The method of claim 22, wherein each sample of the at least one protein is in a plurality of well plates in a well plate array.

25. The method of claim 22, wherein the controller script is configured to control at least one instrument, at least one measurement device, or both in an instrumentation platform for:
dispensing the at least one protein and reagents for stabilizing the at least one protein into each well plate in the well plate array; and
initiating polymerization of the samples in each well plate.

26. The method of claim 25, wherein the controller script is further configured to perform the measurement of the at least one output feature.

27. The method of claim 22, wherein the at least one input feature and the at least one output feature of importance of polymers used to stabilize the at least one protein respectively comprise polymer structural features and polymer functional features for stabilizing the at least one protein.

28. The method of claim 22, wherein the at least one output feature of importance comprises an activity of the at least one protein.

29. The method of claim 22, further comprising updating, by the processor, the library with the at least one measured output feature from the samples of the at least one protein corresponding to polymers in the plurality of polymers in the identified set.

30. The method of claim 22, further comprising retraining, by the processor, the at least one machine learning model by inputting the data for each polymer in the identified set of polymers into the at least one machine learning model and matching the at least one predicted output feature to the at least one measured output feature from the samples of the at least one protein corresponding to polymers in the plurality of polymers in the identified set.

31. The method of claim 22, wherein the at least one machine learning model is a random forest machine learning model.

32. The method of claim 22, wherein the at least one protein is an enzyme.

33. The method of claim 32, wherein the enzyme is selected from the group consisting of horseradish peroxidase (HRP), glucose oxidase (GOx), lipase, Chondroitinase ABC (chABC), cellulase, and lactase.

34. The method of claim 32, wherein the reagents for stabilizing the enzyme comprises four monomers, and wherein the stabilized enzyme comprises four parts corresponding to the four monomers.

35. The method of claim 32, wherein monomers used for stabilizing the at least one protein is selected from the group consisting of Methyl methacrylate (MMA), Butyl methacrylate (BMA), Poly(ethylene glycol) Monomethylether Monomethacrylate (PEGMA), 2-Hydroxypropyl methacrylate (2-HPMA), 2-[(diethylamino)ethyl] methacrylate (DEAEMA), [2-(methacryloyloxy)ethyl] trimethylammonium chloride solution (TMAEMA), 3-Sulfopropyl methacrylate (SPMA), N-[3-(Dimethylamino)propyl]methacrylamide (DMAPMA), and 2-(Dimethylamino)ethyl methacrylate (DMAEMA).

36. A method comprising:
receiving, by a processor, from a user, at least one protein for stabilization using polymers, and at least one input feature and at least one output feature of importance of polymers used to stabilize the at least one protein;
identifying, by the processor, a set of polymers from a library of a plurality of polymers for stabilizing the at least one protein using an output of at least one machine learning model;
wherein the at least one machine learning model outputs at least one predicted output feature for each polymer in the library corresponding to the at least one output feature of importance of polymers used to stabilize the at least one protein when inputting data for each polymer in the library into the at least one machine learning model;
wherein the data for each polymer in the library of the plurality of polymers comprises at least:
(i) features for each polymer, and
(ii) reagents for stabilizing the at least one protein;
receiving, by the processor, at least one measured output feature of the polymer;
determining, by the processor, score to each sample of the at least one protein based on a comparison between the at least one measured output feature of the polymer and the at least one output feature of importance;
wherein a higher score is indicative of a higher match between the at least one measured output feature of the polymer and the at least one output feature of importance of the polymer used to stabilize the at least one protein; and
identifying, by the processor, the samples of the at least one protein with scores higher than a predefined threshold.

37. A system, comprising:
an instrumentation platform comprising at least one instrument, at least one measurement device, or both; and
at least one processor configured to:
receive from a user, at least one protein for stabilization using polymers, and at least one input feature and at least one output feature of importance of polymers used to stabilize the at least one protein;
identify a set of polymers from a library of a plurality of polymers for stabilizing the at least one protein using an output of at least one machine learning model;
wherein the machine learning model outputs at least one predicted output feature for each polymer in the library corresponding to the at least one output feature of importance of polymers used to stabilize the at least one protein when inputting data for each polymer in the library into the at least one machine learning model;
wherein the data for each polymer in the library of the plurality of polymers comprises at least:
(i) features for each polymer, and
(ii) reagents for stabilizing the at least one protein;
generate a controller script for implementing an experimental design flow for stabilizing samples of the at least one protein based on the at least one predicted output feature for each polymer in the identified set;
receive, based on the controller script, a measurement of the at least one output feature of the sample of the at least one protein corresponding to the at least one output feature of importance;
assign a score to each sample of the at least one protein based on a comparison between the at least one measured output feature of the polymer and the at least one output feature of importance;
wherein a higher score is indicative of a higher match between the at least one measured output feature of the polymer and the at least one output feature of importance of the polymer used to stabilize the at least one protein; and
identify the samples of the at least one protein with scores higher than a predefined threshold.

38. The system according to claim 37, wherein each sample of the at least one protein corresponds to each polymer in the identified set of polymers.

39. The system according to claim 37, wherein each sample of the at least one protein is in a plurality of well plates in a well plate array.

40. The system according to claim 37, wherein the controller script is configured to control at least one instrument, at least one measurement device, or both in an instrumentation platform for:
dispensing the at least one protein and reagents for stabilizing the at least one protein into each well plate in the well plate array; and
initiating polymerization of the samples in each well plate.

41. The system according to claim 41, wherein the controller script is further configured to perform the measurement of the at least one output feature.
